(19) United States
(12) Patent Application Publication
(10) Pub. No.: US 2024/0156145 A1
(43) Pub. Date: May 16, 2024

(54) SYSTEMS AND METHODS FOR MANUFACTURING API-INFUSED EDIBLES, AND RESULTING EDIBLE PRODUCTS

(71) Applicant: Transport Authority, Inc., Dover, DE (US)

(72) Inventors: William Warren, Lafayette, CA (US); Walter Lee Smith, Danville, CA (US)

(21) Appl. No.: 18/509,855

(22) Filed: Nov. 15, 2023

Related U.S. Application Data
(60) Provisional application No. 63/383,898, filed on Nov. 15, 2022.

(51) Int. Cl. A23P 20/20; A23G 1/00; A23G 1/48; A23G 1/54; A23G 3/34; A23G 3/48; A23G 3/54

(52) U.S. Cl. CPC A23P 20/20; A23G 1/0006; A23G 1/48; A23G 1/54; A23G 3/0065; A23G 3/48; A23G 3/54

(57) ABSTRACT

This present disclosure provides reliable methods and apparatus for delivering a pharmaceutical active ingredient to an edible substrate. In some examples, the pharmaceutical active ingredient is delivered to voids that are formed in the edible substrate. The voids can then be closed as desired. The edible substrate can be configured as a food product or a dosage form as desired.

### CROSS-REFERENCE TO RELATED APPLICATIONS

This claims priority to U.S. Patent Application Ser. No. 63/383,898 filed Nov. 15, 2022, the disclosure of which is hereby incorporated by reference as if set forth in its entirety herein.

### BACKGROUND

### Field of the Art

The disclosure relates to the field of active pharmaceutical ingredients, and more particularly to the field of edible products comprising active pharmaceutical ingredients, and methods and systems for manufacturing the same.

### Discussion of the State of the Art

The dosed edible industry is growing rapidly in the United States, Canada, and across the world. Methods and apparatus exist for applying active pharmaceutical ingredients (APIs) to edibles for later consumption. For instance, APIs can be delivered in liquid form to the exterior of the edible as sprays, drops, or the like. However, in some instances it may be desirable to infuse the edible with one or more APIs. Conventional methods inject the API-containing liquid directly into the edible. However, it can be difficult to precisely control the quantity of cannabis infused into the edible using such dosing techniques. Another challenge is in ensuring that the delivered liquid remains in the edible without flowing out of the edible during and after the injection process. Even if the liquid ultimately dries on the outer surface of the edible, the taste profile of the edible will be affected when first placed into the oral cavity. It can be desirable for an improved method and apparatus for dosing edibles.

### SUMMARY

In one aspect, a method is provided for delivering an active pharmaceutical ingredient (API) to an edible substrate. The method can include the steps of operably aligning a pre-formed void that extends into the edible substrate with a dosing head, and delivering a volume of at least one API-containing liquid from the dosing head into the void.

In another aspect, a method is provided for creating voids in a food product that are configured to receive API-containing liquid.

### BRIEF DESCRIPTION OF THE DRAWINGS

The following detailed description will be better understood when read in conjunction with the appended drawings, in which there is shown in the drawings example embodiments for the purposes of illustration. It should be understood, however, that the present disclosure is not limited to the precise arrangements and instrumentalities shown. In the drawings:

FIG. 1A is a schematic view of an infusion dosing system in one example, including a void forming station, an infusion dosing station, and a closing station;

FIG. 1B is a schematic view of a substrate that progresses through the stations of the infusion dosing system of FIG. 1A;

FIG. 2A is a schematic view of an opener of the void forming station of FIG. 1, configured as a laser in one example;

FIG. 2B is a schematic view of an opener configured as a drill bit in another example;

FIG. 2C is a schematic view of an opener configured as a displacement shaft in another example;

FIG. 2D is a schematic view of an opener configured as a heated shaft in another example;

FIG. 2E is a schematic view of a substrate having a through-hole configured to define a void for receiving API in one example;

FIG. 2F is a schematic view of the substrate of FIG. 2E having a void closed by a plug inserted into one end of the through-hole;

FIG. 2G is a schematic view of the substrate having a void closed at one end by a layer that extends along an end of the through-hole;

FIG. 3A is a schematic view of an array of openers of the void forming station of FIG. 1, in another example;

FIG. 3B is an enlarged detailed view of the openers of FIG. 3A;

FIG. 3C is a perspective view of an edible substrate having voids created by the openers of FIG. 3A;

FIG. 3D is a schematic sectional view of a mold that creates an edible substrate having voids in one example;

FIG. 3E is a schematic sectional view of a mold that creates an edible substrate having voids in another example;

FIG. 4A is a schematic view of the infusion dosing station of FIG. 1, shown delivering an active pharmaceutical ingredient (API) into a pre-formed void of an edible product;

FIG. 4B is an enlarged schematic view of the edible product of FIG. 4A, shown with the void containing the active pharmaceutical ingredient;

FIG. 5A is a schematic view of the infusion dosing station of FIG. 1 in another example, including a plurality of dosing heads delivering respective APIs into respective pre-formed voids of an edible product;

FIG. 5B is an enlarged schematic view of the edible product of FIG. 5A, shown with the voids containing the respective APIs;

FIG. 5C is a schematic view of an edible product dosed by an infusion dosing station configured to deliver a spray of liquid-containing API;

FIG. 5D is a schematic view of an edible product dosed by an infusion dosing station configured to deliver a stream of liquid-containing API;

FIG. 5E is a schematic view of an edible product dosed by an infusion dosing station configured to deliver a squirt of liquid-containing API;

FIG. 6A is a schematic view of a closing device of the closing station of FIG. 1 in one example, comprising a material delivery member in one example;

FIG. 6B is a schematic view of a closing device configured as a displacement tool configured to close the void after the API has been delivered therein;

FIG. 6C is a schematic perspective view of the edible shown with a void that has been closed using a closing device;

FIG. 6D is a schematic perspective view of a closing device constructed as a heating element configured to close the void after the API has been delivered therein;

FIG. 7A is a perspective view of a non-deformable edible shown as an M&M candy infused with an API in another example;

FIG. 7B is a sectional side elevation view of the candy of FIG. 7A;

FIG. 7C is an enlarged view of voids of the candy illustrated in FIG. 7B, shown infused with an API and open;

FIG. 7D is an enlarged view of voids of the candy illustrated in FIG. 7C, whereby the voids are closed;

FIG. 7E is a sectional side elevation view of the candy illustrated in FIG. 7A, whereby voids are shown containing with an API and shown open;

FIG. 7F is a sectional side elevation view of the candy illustrated in FIG. 7E taken along line 7F-7F of FIG. 7A, showing the voids closed;

FIG. 8A is a schematic perspective view of an elastically deformable edible shown as a gummy candy infused with an API in a plurality of closed voids;

FIG. 8B is a schematic perspective view of an elastically deformable edible shown as a fruit roll-up infused with an API in a plurality of closed voids;

FIG. 8C is a schematic perspective view of an elastically deformable edible shown as Starburst® candy infused with an API in a plurality of closed voids;

FIG. 9 is a perspective view of a dosage form configured to receive respective at least one API-containing liquid using the infusion dosing system of FIG. 1;

FIG. 10A is a schematic cross-sectional view of coating device inserted into a void of an edible substrate so as to create a nonporous barrier at a perimeter of the void;

FIG. 10B is a schematic cross-sectional view of the void of FIG. 10A, shown with the nonporous barrier at the perimeter of the void;

FIG. 11A is a schematic side elevation view of a layered edible substrate including a first active layer, a second active layer, and a partition layer disposed between the first and second active layers in a void of the substrate;

FIG. 11B is a schematic side elevation view of the substrate of FIG. 11A, showing the first active layer disposed in the void;

FIG. 11C is a schematic side elevation view of the substrate of FIG. 11B, shown including the partition layer;

FIG. 12A is a schematic cross-sectional view of an edible substrate having first and second voids that contain respective first and second active pharmaceutical ingredients configured for transmucosal ingestion and gastrointestinal ingestion, respectively; and

FIG. 12B is a perspective view of an edible substrate having groups of voids containing respective APIs.

### DETAILED DESCRIPTION

Disclosed are systems and methods for manufacturing cannabis edibles, and resulting edible products, by creating voids that extend into the edibles, and delivering API-containing liquid into the voids.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

Headings of sections provided in this patent application and the title of this patent application are for convenience only and are not to be taken as limiting the disclosure in any way.

When a single device or article is described herein, it will be readily apparent that more than one device or article may be used in place of a single device or article. Thus, reference to a single device or article can include at least one or a plurality of the devices or articles. Similarly, where a plurality of devices or articles is described herein, it will be readily apparent that a single device or article may be used in place of the plurality of devices or articles. Thus, reference to a plurality of devices or articles can include one or at least one device or article.

The terms “substantial,” “approximate, “about,” derivatives thereof, and words of similar import when used with respect to a quantity, volume, mass, weight, dosage, size, shape, direction, or other parameter, include the stated parameter specifically along with ranges within plus and minus 20% of the stated parameter, for instance plus and minus 10% of the stated parameter, including within plus and minus 5% of the stated parameter, such as within plus and minus 2% of the stated parameter, including plus and minus 1% of the stated parameter.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

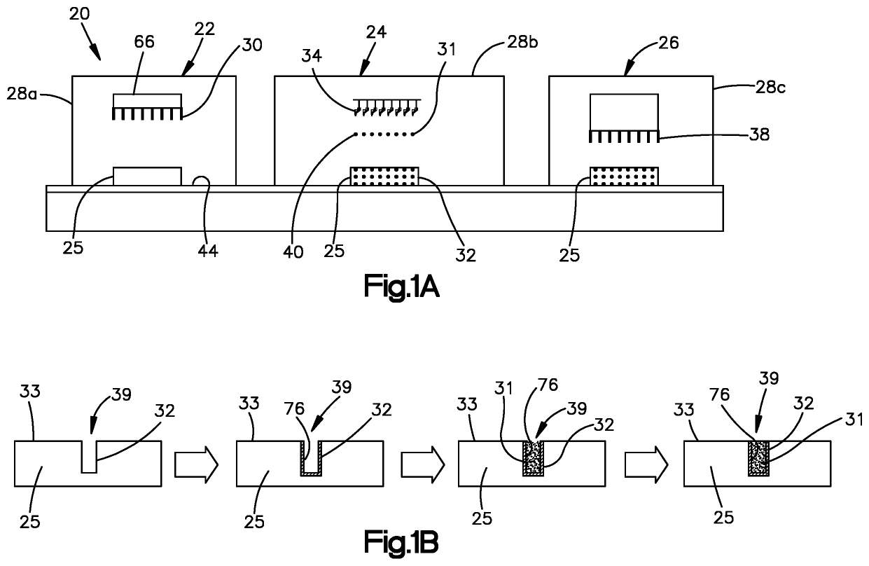

It should be appreciated from the description above that methods and apparatus are provided for transforming an edible substrate, which can be a food product or other edible product, into an accurately and repeatably and precisely dosed cannabis or other API-containing products. As will be described, the edible substrates can be consistently dosed to avoid substantial variations in API dosage levels among a group of edible products or other substrates that are intended to have the same dosage of API. The method can include the step of delivering the API, which can include cannabis including acidic, neutral, and/or emulsified forms of cannabis, or other non-cannabis substances, to the substrate 25 of FIGS. 1A-1B. The delivering step can be performed with any suitable one or more applicators that deliver a predetermined dose of the cannabis. The term “cannabis” as used herein refers to any extract from a marijuana plant or hemp plant, such as CBD, THC, or any alternative cannabinoid, alone or in combination with any one or more of a flavonoid or terpene. The terpenes and/or flavonoids could be extracted from cannabis or hemp, or could be provided as pure substances acquired or synthesized commercially from other sources. The extract can be in its pure form or processed as desired, including as example, emulsified forms of cannabis fluids. While this disclosure provides for the addition of at least one cannabis to a substrate, and thus cannabis is contemplated as a market for the final product, applications of the systems and methods disclosed herein are possible and envisioned that do not involve cannabis, including (but not limited to) other APIs. For instance, applications of the systems and methods disclosed herein are possible and envisioned to include APIs including one or more cannabinoids, any alternative one or more over-the-counter (OTC) or prescription drugs including those that provide one or both of a health benefit or recreational drug experience, otherwise controlled ingestible materials, and herbal medicines, supplements, and the like. Thus, reference herein to an active pharmaceutical ingredient can include any one or more up to all of the following: cannabis and cannabis plant-derived compounds, including one or more cannabinoids in either natural oily forms or emulsified forms, one or more over-the-counter drugs, one or more prescription drugs, one or more flavonoids, one or more terpenes, and one or more herbal medicines or supplements. Reference herein to an API can further include any one or more active ingredients having a medicinal benefit, including prescription medications and over-the-counter medications, further including time-release medications, and further including vitamins, nutritional and longevity supplements, and nootropics or so-called “smart drugs” as desired. Reference herein to an active pharmaceutical ingredient can alternatively or additionally include one or more up to all of the following: psychedelic or hallucinogenic ingredients such as psilocybin and psilocyn, and synthetic opioids including synthetic opioid pain reducers. While accurately dosed synthetic opioids can replace the more dangerous ingredients such as fentanyl, it should be appreciated that the API can alternatively or additionally include fentanyl, recognizing the accurate dosage of the type described herein can be of importance when administering an API that can have severe consequences when inaccurate dosages are administered. Similarly, reference to one or more of the active pharmaceutical ingredients identified above can apply equally to any other of the active pharmaceutical ingredients identified above. According to an aspect of the present disclosure, a method of delivering a cannabis or conventional drug may also be used to deliver homeopathic remedies, herbal supplements, with flavors or odors, and so forth to an edible substrate. The resulting edible substrate can be referred to as a “nutraceutical,” as its definition is “a food containing health-giving additives or having medicinal benefit.” Examples of such APIs include any one or more APIs commercially available from Lost Empire Herbs having a principal place of business in Kansas City, MO, including Thor's Hammer Capsules, Phoenix Rejuvenation Formula, Stag Swag, Spartan Formula, Athena, Hercules, Mushroom Alchemy Tincture, Goji Schisandra Tincture, Chiron Immunity Tincture, Pine Pollen Capsules, Pine Pollen Tincture, Blue Vervain Spagric Tincture, Ashwagandha Spagyric Tincture, Pine Pollen Tincture, Nettle Root Tincture, Tongkat Ali, Pine Pollen, Megadose, Nettle Root, Shilajit Powder, Shilajit Resin, Lion's Mane, Mushroom Alchemy Tincture, Cordyceps, Reishi, Chaga, Nettle Root Spagyric Tincture, Elderberry Extract, Goji & Schisandra Tincture, Chiron Immunity Tincture, Schisandra Berry, Goji Berry, Sea Buckthorn, He Shou Wu, Cistanche, Black Ant, Black Maca, Mucuna, Horny Goat Weed, Bacopa, Beet Juice Powder, Pearl Powder, Albizia, Gynostemma, Ziziphus, and Rhodiola. Examples of such APIs can further include any one or more of Dynamic Brain commercially available by Stonehenge Health having a principal place of business in Newport Beach, CA, Qualia Mind commercially available from Neurohacker having a principal place of business in Carlsbad, CA, Genius Consciousness commercially available from The Genius Brand having a principal place of business in Buford, GA, Neuriva Brain Performance commercially available from Schiff Vitamins having a principal place of business in Salt Lake City, UT, and Cerebra commercially available from Boston Brain Science having a principal place of business in Lakeway, TX. Examples of such APIs can further include one or more of ginseng, sage, guarana, bacopa monnieri, peppermint, rosemary, rhodiola rosea, ashwagandha, centella sciatica, maca, gotu kola, caffeine, L-theanine, creatine, bacopa monnieri, rhodiola rosea, panax ginseng, ginkgo biloba, nicotine, noopept, piracetam, phenotropil, modafinil, amphetamines, methylphenidate, lysine, tarragon, vitamin E, zinc, Vitamin D, garlic, spinach, beets, kale, spinach, and broccoli.

Referring to FIGS. 1A-1B, an infusion dosing system 20 is configured to create one or more voids 32 in an edible substrate 25, and deliver at least one API-containing liquid into the one or more voids 32, thereby infusing the API into the edible substrate 25. The voids 32 can define an opening 39 at a first surface of the substrate 25, and can extend toward but not through an opposed second surface of the substrate 25.

It will be appreciated that the edible substrate 25 can be configured as a food product having nutritional value. That is, the edible substrate 25 can be configured as a food product that is typically consumed by humans even when the edible substrate 25 is not dosed with an API. This is in contrast to dosage forms such as pills, tablets ,and capsules that include an excipients for the purpose of delivering an API. Dosage forms having excipients without an API have little to no nutritional value, and are not typically consumed by humans. Thus, dosage forms do not qualify as food products. In one example, a food product can have a non-trivial amount of calories, such as at least approximately 3, at least approximately 4, at least approximately 5, at least approximately 6, at least approximately 7, at least approximately 8, and at least approximately 9 calories. For instance, in one example, a conventional gummy bear has approximately 9 calories. The calories can comprise at least one or more up to all of protein, fat, carbohydrates, and alcohol. In another example, a food product can include a sweetener. In other examples, a food product can include a flavoring agent. In still other examples, a food product is not in a capsule, tablet, or pill form.

While such dosage forms contain desired quantities of API for human consumption, the consumption of dosage forms in the past has resulted in catastrophes, such as the sulfanilamide mass poisoning of over 100 people in 1937, and the thalidomide deformities of over 10,000 babies worldwide in the 1950's-1960's. Each of these has contributed to the current rigorous level of FDA testing. While the general populace has today become accustomed to ingesting such dosage forms and medicines in general, the APIs of such dosage forms are typically carried by excipients that lack nutritional value, may trigger immune system reactions, and may not have undergone testing for potential harmful interactions with other API's being consumed by a given patient.

As will be appreciated from the description below, one or more APIs can be infused into a food product suitable for human consumption. Advantageously, the food product lacks excipients, and the one or more APIs is delivered by the food product itself. Such excipient-free API delivery systems can overcome the potential disadvantages associated with excipients as described above. Further, the use of food product as a delivery vehicle for APIs allows a user to determine a particular food product that is desired for consumption in order to ingest the desired API or APIs. Further, because the food product can be dosed with several APIs as will be described below, the consumer has a greater selection of the APIs to be ingested by consuming a single food product a daily basis, rather than the tiring inconvenience of organizing and swallowing several separate dosage forms once or more each day.

In one example, the infusion dosing system 20 can include a void forming station 22, a dosing station 24, and a closing station 26. One or more up to all of the void forming station 22, a dosing station 24, and a closing station 26 can be supported by a respective support structure 28a, 28b, and 28c. Any two or more up to all of the support structures 28a-28c can be separate from each other, joined to each other, or monolithic with each other so as to define a unitary support structure as desired.

With continuing reference to FIGS. 1A-1B, the void forming station 22 can include at least one opener 30 that is configured to create at least one void 32 such as a plurality of voids 32 in an edible substrate 25. Each void 32 can be defined at one end by an opening 39 at a first or outer surface 33 of the edible substrate 25. The opening 39 can thus define an opening to the void 32. The void 32 can be created by the opener 30. The void 32 can extend from the opening 39 or the first surface 33 into the edible substrate 25 along a central axis 46. The void extends from the opening 39 toward a second surface 45 that is opposite the first surface 33, but in one example does not extend through the second surface 45. Thus, each void 32 can be defined by a respective at least one internal surface 35 of the edible substrate 25, and can terminate at a base 37 that is opposite the opening 39. The base 37 can be defined by a region of the edible substrate 25 that is opposite the opening 39 at the outer surface 33.

The dosing station 24 can include at least one dosing head 34 such as a plurality of dosing heads 34 that are configured to deliver at least one active pharmaceutical ingredient (API) containing liquid 31 into the at least one void 32. In one example, the API-containing liquid 31 can be delivered as microdroplets 40 described in more detail below. Once the API-containing liquid 31 has been delivered to the edible substrate 25, the edible substrate 25 can be referred to as a dosed edible substrate, or dosed edible such as a dosed food product. The closing station 26 can include a closing device 38 that is configured to close the void 32. For instance, the closing station 26 can close the opening 39 so as to enclose the void 32. In some examples, the closing station 26 can be optional, for instance when the API-containing liquid 31 is absorbed from the voids 32 into the edible substrate 25. The infusion dosing system 20 can also include a packaging station as desired, which can be configured to secure one or more edible substrates in packaging, which may be air-tight, or oxygen-free, or nitrogen backfilled, for shipping.

The active pharmaceutical ingredient can be added to the edible substrate 25 to produce an active-containing substrate. In one example, the edible substrate can be a food product having nutritional value to which the API can be added to produce an active-containing food product. The food product can be configured to be masticated and/or swallowed. Alternatively or additionally, the food product can be configured to be dissolved in the mouth.

It is recognized that the edible substrate 25 can be porous or non-porous. When the edible substrate 25 is non-porous, the API-containing liquid 31 can remain in the voids 32 without migrating from the voids into the substrate material. In other examples, the edible substrate 25 is porous. In some embodiments, prior to delivering the API-containing liquid 31 to the voids 32, a non-porous barrier 76 can be applied to the edible substrate 25 in one or more up to all of the voids 32 as desired, so as to prevent migration of the API-containing liquid from the voids 32 into pores of the edible substrate 25 when the edible substrate 25 is porous. Thus, when the API-containing liquid is delivered into the voids 32, the API-containing liquid 31 will remain in the voids 32.

Edible substrates can include food products that, in turn, can include by way of example and not limitation a prepared food product such as hard candy, chocolates, brownies, cake, cookies, soft candies such as gummy candy, taffy, tootsie roll, Starburst® candy, licorice, fruit roll-up, savories such as trail mix bars or dried meat pieces, and the like. Thus, in some examples, the prepared edible food product can be cooked food product. In some specific examples, the prepared edible substrates can be baked food product. The prepared food product can be bite sized, such as M&M candy, gummy candies, chocolate kisses, or the like, or can be designed to require more than one bite for full consumption, such as a cookie, brownie, or cake. Thus, in some examples, the prepared food product can include a plurality of mixed ingredients. In some examples, the prepared food products can be a dehydrated food product, such as dried fruit or jerky. In other examples, the prepared food product can be freeze dried. In still other examples, the prepared food product can be a raw or uncooked food product, such as nuts or fruit. It will thus be appreciated that the application of the active pharmaceutical ingredient to the previously prepared food product can allow for a broader range of food product to be made with active pharmaceutical ingredients. It will be appreciated that because the active pharmaceutical ingredients are applied to previously prepared food product, active pharmaceutical ingredients having short shelf lives can be applied to the substrate and ingested in a shorter period of time with respect to active pharmaceutical ingredients that are combined with the raw ingredients that are then processed to prepare the food product. While certain food products have been described, it should be appreciated that the present disclosure is intended to apply to any suitable food product as desired.

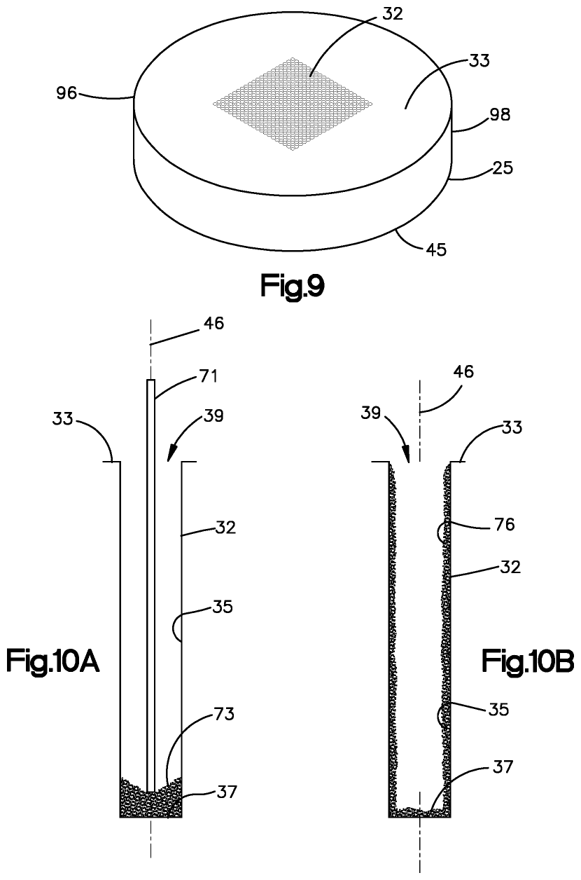

Further, while descriptions herein pertain to one or more edible substrates in the form of food products having nutritional value, it should be appreciated that the description can apply with equal force and effect to any edible substrate regardless of whether the substrate has nutritional value. In some examples, the edible substrate can have no or minimal nutritional or caloric value, but is configured for human consumption. One such example is a medicinal dosage form to which the API can be added to produce an active-containing medicinal dosage form 96 (see FIG. 9). The medicinal dosage form can be configured as tablet, pill, or can have any suitable configuration as desired.

In one aspect, a plurality of active-containing edible substrates can be provided as a set, wherein some of the substrates of the set have different dosages of the active pharmaceutical ingredients and are designed to be ingested at different times among a period of time, such as different days of the week. Thus, ingesting predetermined substrates of the set at predetermined times can cause a desired dosage profile to be delivered to the patient over a period of time. Alternatively or additionally, one of the active-containing substrates can contain a different at least one active pharmaceutical ingredient with respect to at least one other of the active-containing substrates of the set. Thus, the set of active-containing substrates can be designed to be sequentially ingested (that is, ingested one after the other) over the period of time, thereby delivering a desired predetermined sequence of active pharmaceutical ingredients to the patient.

The API-containing liquid 31 can be in the form of a pure API, such as a resin, or can be in the form of a concentration of API in a liquid carrier such as a solvent. In some examples, any suitable applicator such as the dosing head 34 can deliver quantities of the API-containing liquid 31 to the substrate 25.

The infusion dosing system 20 can be configured to transport a plurality of edible substrates 25 to the void forming station 22, from the void forming station 22 to the dosing station 24, and from the dosing station 24 to the closing station 26. In one example, the infusion dosing system 20 can include at least one support member that defines a respective at least one support surface 44 that is configured to support the edible substrates 25. At least one or more of the support surface 44, the openers 30, the dosing heads 34, and the closing device 38 can be movable so as to bring the edible substrates 25 into proper alignment with the respective stations of the infusion dosing system 20.

In one example, the support surface 44 can be configured as any suitable delivery member such as a conveyor or other suitable support member that is designed to support and transport the edible substrates 25 to be brought into operable alignment with a respective one of the void forming station 22, the dosing station 24, and the closing station 26. Alternatively, the support surface 44 can be stationary, at least one or more of the support surface 44, the openers 30, the dosing heads 34, and the closing device 38 can be movable. Alternatively still, both the support surfaces 44 and at least one of the openers 30, the dosing heads 34, and the closing device 38 can be movable so as to bring the edible substrate 25 into alignment with the dosing heads of the dosing station 24.

Alternatively still, a user can place the substrate 25 onto the support surface 44 at one or more up to all of the void forming station 22, the dosing station 24, and the closing station 26. The user can manually place the substrate 25 or can use any suitable automated technology such as pick-and-place. In this example, the support surface 44 and at least one or more up to all of the void forming station 22, the dosing station 24, and the closing station 26 can be stationary.

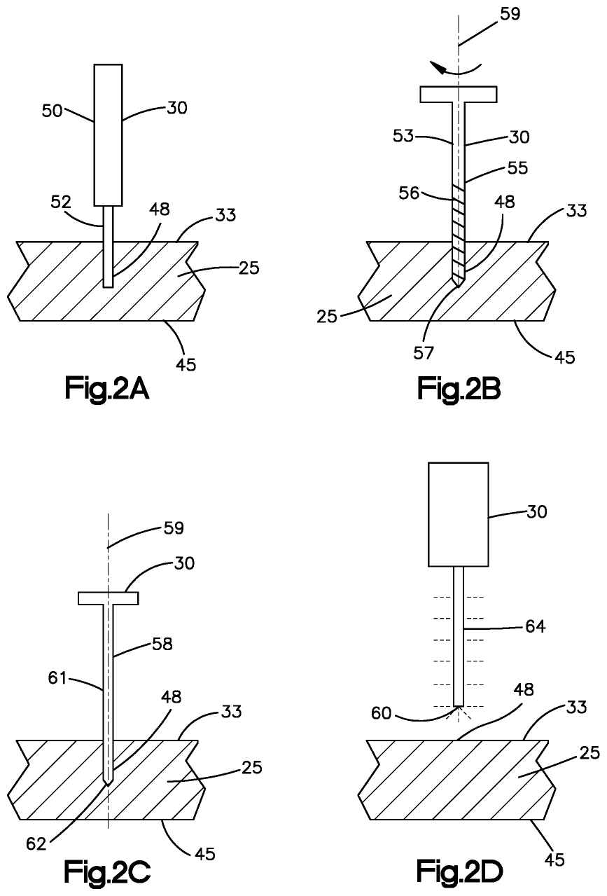

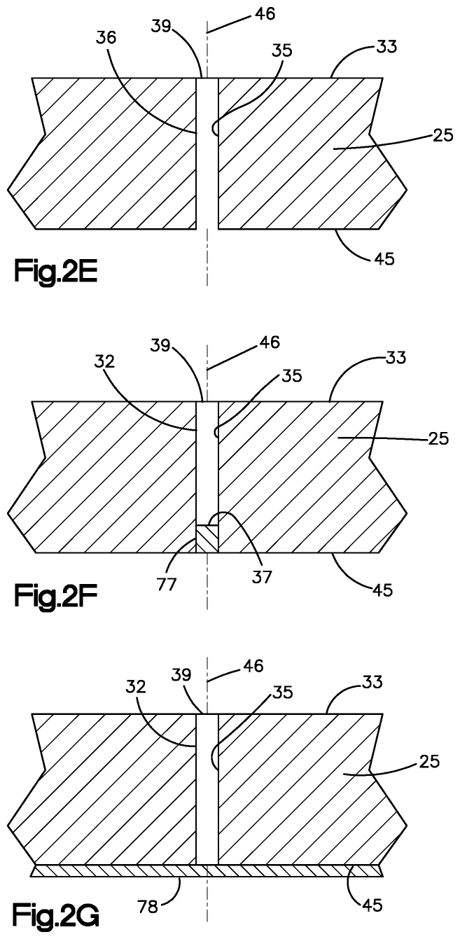

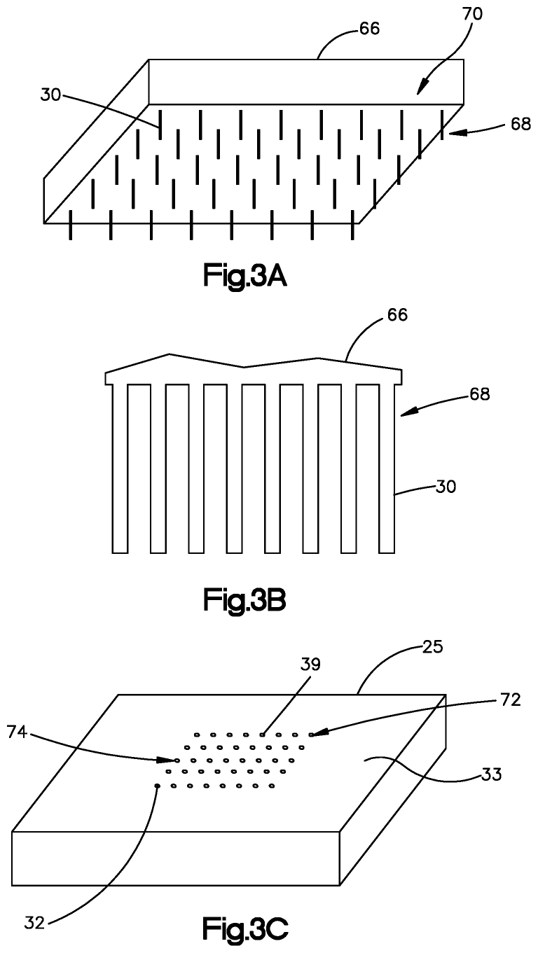

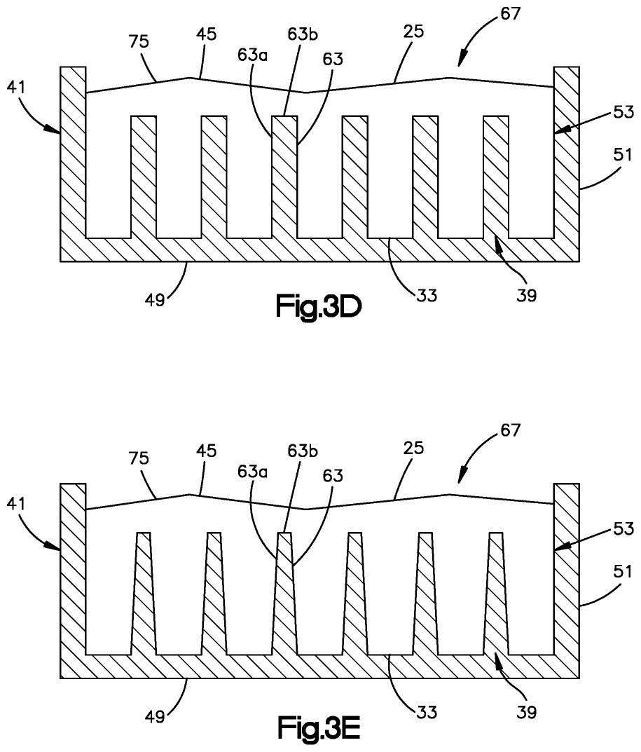

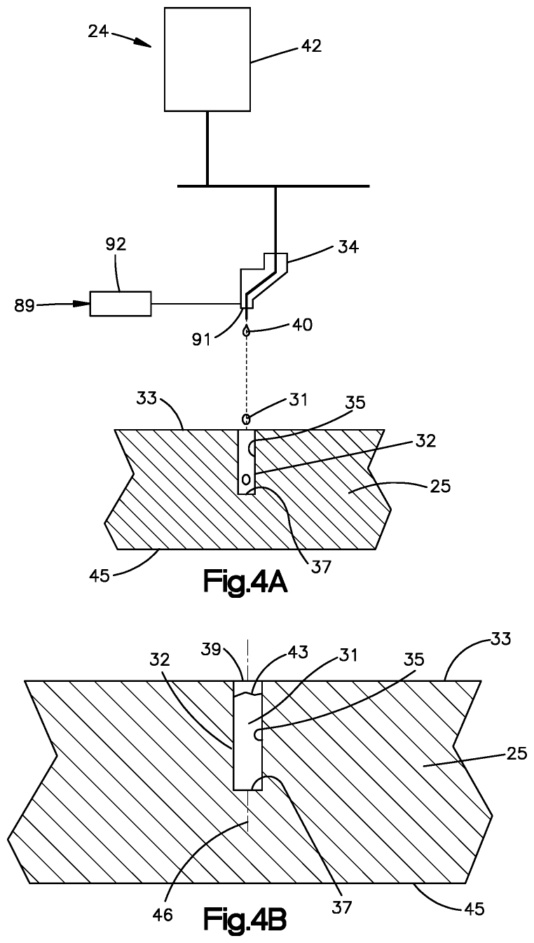

Each station of the infusion dosing system 20 will now be described, with initial reference to FIGS. 2A-3C. In particular, when the edible substrate 25 is aligned with the void forming station 22, the at least one opener 30 is configured to create the voids 32 in the substrate 25. The opener 30 can be configured to create the voids 32 in any suitable manner as desired. Each void 32 extends into the substrate 25 along a central axis 46 from the first surface 33 toward but not through the second surface 45 to a base 37 (see FIGS. 4A-4B) along a dimension that defines the depth of the void. Each void 32 can define a maximum cross-sectional dimension along a direction perpendicular to the central axis 46. In some examples, the void 32 can define a shaft when the depth is greater than the maximum cross-sectional dimension. It is recognized, of course, that the void 32 can have any suitable size and shape as desired.

The maximum cross-sectional dimension is sufficiently large to receive the API-containing liquid 31 in any suitable manner as described herein. In one example, the maximum cross-sectional dimension of each void 32 along a direction perpendicular to the central axis 46 is therefore greater than an outermost dimension of the API-containing liquid as it is delivered into the void 32. For instance, as described above, the API-containing liquid 31 can be delivered as drops, microdroplets, a stream, spray, or any suitable manner as desired. Alternatively, while the API can be delivered as an API-containing liquid 31 in the manner described herein, the API can alternatively be delivered in dry form, for instance as dry particulates such as a powder or other dry form as desired. The maximum cross-sectional dimension can be sufficient such that the void 32 is sized to receive the API-containing liquid 31 through the opening 39 and vent out the opening 39 the gas, such as air, that was disposed in the void 32 and displaced by the API-containing liquid 31 that is delivered into the void 32.

In certain examples, the maximum cross-sectional dimension can be in the range of 5 millionths of an inch to approximately 0.25 inch. For instance, the range can be from approximately 5 millionths of an inch to approximately 100 thousandths of an inch, such as from approximately 5 thousandths of an inch to approximately 50 thousandths of an inch. In one example, the maximum cross-sectional dimension along the select direction can be in a range from approximately 20 thousandths of an inch to approximately 40 thousandths of an inch. However, it should be appreciated that the maximum cross-sectional dimension can be any suitable dimension as desired. In one example, the maximum cross-sectional dimension can be up to approximately 10% of the dimension of the edible substrate 25 along a common direction, so that the void 32 does not reduce the structural integrity of the edible substrate 25. In one example, the void 32 can be substantially cylindrical, such that the maximum cross-sectional dimension is a diameter of the void. It should be appreciated, however, that the voids 32 can have any suitable size and shape as desired.

Referring now to FIG. 2A, the opener 30 can be configured to remove at least one select region 48 of the edible substrate 25 so as to define the void 32. For instance, the opener 30 can be configured to ablate the at least one region of the edible substrate 25. In one example the opener 30 can be configured as a laser 50 configured to emit a laser beam 52 having sufficient power to ablate the at least one region 48 so as to create the void 32. The laser beam 52 can be configured to sequentially remove a plurality of regions 48 of the edible substrate 25 so as to create a respective corresponding plurality of voids 32 as desired. Alternatively, as will be described in more detail below, the void forming station 22 can include a plurality of lasers 50 arranged in any pattern as desired so as to create a respective array of voids 32 arranged in the pattern.

Referring to FIG. 2B, in another view the opener 30 can be configured as a drill bit 54 configured to remove the at least one region 48 of the edible substrate 25. The drill bit 54 include a shaft 53 that defines an outer surface 55 having at least one cutting flute 56. The shaft 53 further defines a distal end 57 that can be tapered so as to define a tapered tip, or can alternatively be blunt as desired. The distal end 57 can define a leading end with respect to insertion of the drill bit 54 into the edible substrate 25. The cutting flute 56 can be helical or alternatively shaped. The at least one cutting flute 56 can be configured to create the void 32 when the drill bit 54 is driven into the edible substrate 25. In particular, the drill bit 54 can be rotated about its central axis 59 as it is driven into the edible substrate 25 from the first surface 33, such that the at least one cutting flute removes the region 48 of the edible substrate 25. The drill bit 54 can be supported by an automatic drill that applies a rotational force to the drill bit 54 that causes the drill bit to rotate about its axis. Alternatively, the drill bit 54 can be manually rotated as desired.

Referring to FIG. 2C, in another view the opener 30 can be configured as a displacement shaft 58 that can be configured to create the void 32. The displacement shaft 58 can be elongate along a central axis 59, and can be driven along the central axis 59 into the edible substrate 25, and in particular into the region 48. The displacement shaft 58 defines an outer surface 61 and a distal end 62. The distal end 62 can be tapered so as to define a tapered tip, or can be blunt as desired. The distal end 62 can define a leading end with respect to insertion of the displacement shaft 58 into the edible substrate 25. In one example, the displacement shaft 58 can be translated without substantial rotation into the edible substrate 25. “Substantial” in this context can mean that the translation would be sufficient to create the void 32 without the rotation. In some examples, without substantial rotation can mean less than three revolutions about the central axis 59, such as less than two revolutions about the central axis 59, for instance less than one revolution about the central axis 59. Alternatively, the displacement shaft 58 can be rotated as it is translated. As the displacement shaft 58 is driven into the region 48 from the first surface 33, the displacement shaft 58 causes the edible substrate 25 in the region 48 to be displaced so as to create the void 32. At least some up to all of the edible substrate 25 in the region 48 can be compressed by the displacement shaft 58 as the displacement shaft 58 is driven into the edible substrate 25 so as to create the recess 32. In some examples, the outer surface 61 of the shaft 58 can be smooth along an entirety of the outer surface 61 that is driven into the edible substrate 25. Alternatively, the outer surface 61 of the shaft 58 can be textured as desired.

Referring now to FIG. 2D, the opener 30 can be configured as a heated shaft 64. The shaft 64 can carry embedded heating coils, or can otherwise be raised to a temperature greater than ambient temperature. In particular, the heated shaft 64 can emit heat 60 so as to define the raised temperature along at least a portion of the heated shaft 64 that is to be driven into the region 48 so as to selectively melt the edible substrate 25 in the region 48. In one example, for instance when the edible substrate 25 is in a solid state, the temperature of the heated shaft 64 can be sufficient to cause the edible substrate 25 in the region 48 to melt and liquify, thereby defining the void 32. In another example, for instance when the edible substrate 25 is in a viscous state, the temperature of the heated shaft can be sufficient to cause the edible substrate 25 in the region to melt, thereby lowering the viscosity of the edible substrate 25 and causing the edible substrate 25 to flow out of the region 48 and create the void 32. The heated shaft 64 can be constructed as described above with respect to the drill bit 54. In another example, the heated shaft 64 can be constructed as described above with respect to the displacement shaft 58, or can alternatively be constructed as desired.

As described above, the base 37 can be defined by the edible substrate 25 that is opposite the opening 39 at the outer surface 33. In other examples, as shown at FIG. 2E, the void 32 can be created from a hole 36 that extends entirely through the substrate 25 from the first surface 33 to the second surface 45. Thus, the hole 36 defines respective first and second open ends at the first and second surfaces 33 and 45, respectively. The second open end of the hole 36 can be closed. For instance, as shown at FIG. 2F, any suitable edible plug 77 can be inserted into the second end of the hole 36 so as to close the second end of the hole. The plug 77 can therefore define the base 37 of the void 32. Accordingly, the plugged hole 36 can define the void 32 that extends into but not through the substrate 25. Alternatively, as shown at FIG. 2G, any suitable edible layer 78 can extend at least partially along the second surface 45 and across the second open end of the hole 36, thereby closing the second open end of the hole 36. The layer 78 can therefore define the base 37 of the void 32. Thus, in this manner, the plugged hole 36 can define the void 32 that extends into but not through the substrate 25.

Referring now to FIGS. 3A-3C, the void forming station 22 can include a single opener 30 that can create individual voids as described above, or the void forming station 22 can alternatively include a plurality of openers 30 that are each configured to create a plurality of voids 32 in the edible substrate 25. Thus, the step of driving the opener 30 into the edible substrate 25 can therefore include driving a plurality of openers 30 into the edible substrate 25 at a corresponding plurality of select regions 48 (see FIGS. 2A-2D) of the edible substrate 25. The openers 30 can be configured in any manner described above, or alternative opener suitable to create the voids 32 in the edible substrate 25 as desired. In one example, the void forming station 22 can include a forming base 66, and a plurality of openers 30 that extend from the opener base 66.

The openers 30 can thereby create a plurality of voids 32 in the edible substrate. In particular, the respective openers 30 can be inserted or driven into the edible substrate 25 at a plurality of respective select locations or regions 48 so as to create the plurality of voids 32. In one example, the openers 30 can be defined by lasers of the type described above. The lasers can substantially simultaneously ablate the regions 48 of the edible substrate 25 so as to create the voids 32, or can ablate the regions 48 at different times as desired. In another example, the openers 30 can be configured as shafts described above that can be driven into the edible substrate 25 simultaneously so as to create the plurality of voids 32. The central axes of the openers 30 can be oriented substantially parallel to each other, or can be angularly offset from each other as desired. The openers 30 can create respective voids 32 that terminate at the same depth in the edible substrate 25, or terminate at different depths in the edible substrate 25. Further, the openers can create respective voids 32 having the same cross-sectional shape and dimension, or having a different cross-sectional shape and/or dimension. Thus, the resulting voids 32 can each have a respective capacity that is configured to receive the same volume of API-containing liquid or different volumes of API-containing liquid.

The openers 30, and thus the resulting voids 32, can be arranged in an array 69 of respective rows 68 and columns 70. The openers 30 along each row 68 can be substantially equidistantly spaced from each other, or variably spaced from each other as desired. Further, the rows 68 can be equidistantly spaced from each other or variably spaced from each other as desired. The openers 30 of the rows 68 can be aligned with each other so as to define columns 70 of openers 30. Alternatively, the openers 30 of the rows can be staggered or offset with respect to the openers 30 of adjacent rows 68. Because the openers 30 are configured to create the voids 32, it should be appreciated that the resulting voids 32 can be arranged in an array 72 that is configured as described above with respect to the array 69 of openers 30. For instance, the voids 32 can be elongate along respective central axes that amre defined by the central axes of the openers 30, particularly when the openers 30 are configured as shafts that extends along respective central axes as described above. As is described in more detail below, the voids 32 can be disposed in different dosing zones 74 that are composed of different ones or more of the voids 32. The voids 32 of each dosing zone 74 can be configured to receive either or both of a different quantity of API-containing liquid than at least one other of the dosing zones 74 and/or can be configured to a different API-containing liquid than at least one other of the dosing zones 74.

While FIGS. 2A-3C show methods of creating the voids 32 in an existing edible substrate 25, in other examples the edible substrate 25 can be fabricated to include the voids 32. For instance, referring now to FIGS. 3D-3E, the constituent components of the edible substrate can be introduced into a mold 41 in a viscous state, and subsequently cooled to define the voids 32. Thus, it should be appreciated in some examples that the voids 32 can be created after the edible substrate 25 has been fully prepared. Thus, the voids 32 are not created during the preparation of the edible substrate 25, but rather after preparation of the edible substrate 25. In other examples the voids 32 can be created during the cooling of a prepared edible substrate 25. Thus, the voids 32 can be prepared after mixing the constituent components of the prepared edible substrate 25. The voids 32 can be created before or after cooking the prepared edible substrates 25. Further, the voids 32 can be created before or after cooling the prepared edible substrates 25.

The mold 41 can include a mold base 49, at least one side wall 51 that extends out from the mold base 49 so as to define a mold cavity 53, and at least one or more mold pillars 63 that project out from the mold base 49 in the mold cavity 53. The pillars 63 can define at least one outer surface 63a that extends from the mold base 49 to a tip 63b. The at least one side wall 51 can extend out from the mold base 49 a distance greater than the distance that the mold pillars 63 extend form the mold base 49. The mold cavity 53 can define an open end 67 opposite the mold base 59. In one example, the cross-sectional dimension of the mold pillars 63 can be substantially constant along a majority or entirety of their lengths as shown in FIG. 3D. In other examples, referring to FIG. 3E, the mold pillars 63 can be inwardly tapered as they extend in a direction away from the mold base 49. For instance, the mold pillars 63 can be inwardly tapered from the mold base 49.

The constituent material 75 of the edible substrate 25 in a viscous state can be introduced through the open end 67 and into the mold cavity 53. The portion of the constituent material 75 that abuts the mold base 49 can define the first or outer surface 33 of the edible substrate 25. The pillars 63 can extend into the constituent material 75. The at least one outer surface 63a of each pillar 63 can define the at least one internal surface 35 of the resulting void 32, and the tip 63b of each pillar 63 can define the base of the resulting void 32. The constituent material 75 can harden in the mold cavity 53 so as to define the edible substrate 25. The edible substrate 25 can then be removed from the mold cavity 53, such that the volume in the edible substrate 25 occupied by the mold pillars 63 define the voids 32. Because the cross-sectional dimensions of the mold pillars 63 can be substantially constant along a majority or entirety of their respective lengths as shown in FIG. 3D, the resulting voids 32 can likewise have a substantially constant cross-sectional dimension along a majority or entirety of their respective lengths. Alternatively, because the cross-sectional dimensions of the mold pillars 63 can taper inwardly as they extend from the mold base 49 as shown in FIG. 3E, the resulting voids 32 can likewise taper inwardly as they extend from the mold base 49 to the base 37 of the void 32.

In one example shown at FIGS. 4A-4B, the quantities of API-containing liquid 31 can be configured as microquantities. Each microquantity of API-containing liquid 31 can be delivered by the applicator as a respective microdroplet 40 each having a volume in a range from approximately 2 nanoliters to approximately 10 microliters, such as from approximately 25 nanoliters to approximately 2 microliters. For instance, the microdroplets 40 can have a volume that is in a range from approximately 25 nanoliters to approximately 1 microliter. The microdroplets 40 can have a concentration of API as desired. For instance, the concentration of API can range from approximately 50 micrograms per microliter of solution to approximately 1 milligram per microliter of solution. In other examples, the API-containing liquid 31 can be a pure resin of the API. The dosing station 42 can include any suitable vision system 89 that can guide alignment of the dosing head 34 with the opening 39 to the respective void 32 along a direction along which the central axis 46 is oriented. The direction can be a vertical direction, such that the opening 39 to the void 32 is operably aligned with the dosing head 34 along the direction of gravitational forces. In one example, the dosing head 34 can be disposed on the central axis 46. The vision system 89 can be configured as desired, and in one example can include any suitable data acquisition member 91 such as a camera or any suitable alternative sensor that communicates with a processor 92, and in particular can provide data regarding the relative position of the dosing head 34 and the void 32, and in particular the opening 39 in some examples. The processor can send movement command signals to one or more actuators that causes either or both of the dosing head 34 and the edible substrate 25 to be automatically moved into operable alignment with each other, such that API dispensed from the dosing head 34 will enter the void 32. The data acquisition member 91 can confirm that the opening 39 to the void 32 and the dosing head 34 are operably aligned, or can continue to send movement command signals until the opening 39 to the void 32 and the dosing head 34 are in operable alignment. Once the opening 39 to the void 32 and the dosing head 34 are operably aligned, the dosing head 34 can deliver the API to the substrate 25 in any manner described herein. The dosing head 34 can be prevented from delivering API to the substrate 25 until it has been confirmed, for instance by the processor 92, that the dosing head 34 is in operable alignment with the opening 39 to the void 32.

The microdroplets 40 can have any suitable size and shape as desired. In one example, the microdroplets 40 can including microquantities of the active pharmaceutical ingredient. For instance, the microdroplets 40 can define a maximum cross-sectional dimension along a horizontal direction that is in a range from approximately 5 millionths of an inch up to approximately 100 thousandths of an inch. For instance, the range can be from approximately 5 thousandths of an inch to approximately 50 thousandths of an inch. In one example, the maximum cross-sectional dimension along the select direction can be in a range from approximately 20 thousandths of an inch to approximately 40 thousandths of an inch.

It is envisioned in one example that applicators delivering like dosages of API to like substrates 25 can deliver approximately the same sized microdroplets 40 to the like substrates 25. Thus, for instance when delivering the API to food products designed to have the same dosage of API, the dosing heads 34 can deliver the approximately same volume and number of microdroplets 40 to each edible substrate, or to a group, such as a serving, of edible substrates.

The dosing heads 34 can be provided that are configured to deliver microdroplets 40 of any suitable volume, such as the volumes described above. Therefore, each microdroplet 40 can contain a microquantity of API in a range from approximately 0.1 micrograms to approximately 10 milligrams, such as from approximately 1 milligram to approximately 2 milligrams. It is recognized, however, that the microdroplets 40 can have any volume as desired. Further, It is recognized that each microdroplet 40 can contain different quantities of API depending, for instance, on the volume of the microdroplet 40. The microquantity of API in a microdroplet 40 can allow the dosage of API delivered to an edible substrate to be precisely controlled. For instance, the respective volumes of microdroplets 40 can be delivered to a substrate within a range from approximately 1% to approximately 10%, for instance approximately 5%, from a target volume of microdroplets 40 at three sigma (i.e., within three standard deviations from the target volume). Thus, the dosage of API in each microdroplet 40 can be delivered to the substrate within a range from approximately 1% to approximately 10%, for instance approximately 5%, from a target dosage at three sigma. The target volume of microdroplets 40 can vary based on the surface area or volume of the substrate to which the microdroplets 40 are to be applied. Similarly, the dosage of API to be delivered to a substrate can likewise be within a range from approximately 1% to approximately 10%, for instance approximately 5% at three sigma (i.e., within three standard deviations) from a target dosage of API to the substrate or a serving of substrates. The target volume of microdroplets 40 can vary based, for instance, on the volume of the voids 32 into which the microdroplets are delivered.

Thus, in one example, the API-containing liquid 31 can be delivered from the dosing heads 34 into the voids 32 in the form of one or more microdroplets 40. Each microdroplet 40 can contain a microquantity of API. Because each microdroplet 30 can have a precise dosage of API, the delivery of microdroplets 40 into the voids 32 may allow for the administration of a precise dosage administration of the API compared to conventional techniques. In one example, the edible substrate 25 is substantially non-porous, and the API-containing liquid 31 remains in the respective void 32 and defines a liquid level 43 that can remain substantially constant. That is, the API-containing liquid 31 is unable to travel through the internal surface and base 37. In other examples, the edible substrate 25 can be porous, and the API-containing liquid 31 travels out of the void 32 as it diffuses through the internal surface 35 and/or base 37, and into the edible substrate 25. The API-containing liquid 31 can occupy a portion of the respective void 32, such as a majority of the respective void 32 up to a substantial entirety of the respective void 32.

Because the microdroplets 40 are delivered into the voids 32, a higher percentage of the API remains with the edible as opposed to conventional techniques that deliver API-containing liquid to an outer surface of the edible substrate 25, which can run off the substrate, be rubbed off of the substrate, or otherwise inadvertently removed from the substrate, particularly when the outer surface of the edible substrate 25 is nonporous. Further, because the API-containing liquid is disposed in the substrate 25 and not on the substrate 25 in some examples, the user will experience the immediate taste of the substrate 25 upon placement of the substrate 25 into the oral cavity. When API-containing liquids are applied to an outer surface of the substrate 25 in accordance with conventional techniques, the user can experience the immediate taste of the API upon placement of the substrate into the oral cavity.

When the API contains cannabis, the API can be a pure cannabis extract in one example. In other examples the cannabis extract can be mixed or otherwise combined with one or more other materials as desired, such as a solvent to produce a liquid in the form of a solution having an approximate concentration of the cannabinoid or other active pharmaceutical ingredient (API) described above. The microdroplets can include a solution that includes the at least one cannabinoid in its liquid form as the solvent mixed with any suitable solute. In order to assist in achieving predictable doses of the at least one cannabinoid, the at least one cannabinoid can be substantially homogeneously mixed with the solute. Alternatively, the microdroplets 40 can consist of or consist essentially of the cannabinoid extract, either purified, partially purified, or unpurified, in liquid form having a desired viscosity that allows for the cannabinoid extract to be reliably dispensed. In some examples, the liquid can be heated to achieve the desired viscosity without mixing the cannabinoid extract in a solute. In some examples, the microdroplets 40 can have an oily or hydrophilic nature. Because the concentration of the cannabinoid in the liquid can be known, a volume of liquid can be predetermined and delivered to the substrate to achieve a desired predetermined approximate dose of the cannabinoid.

The API-containing liquid 31 delivered to the edible substrate 25 can include a single desired API. Thus, the single desired API can be delivered to the edible substrate 25. Alternatively, a plurality of different API-containing liquids 31 can be delivered to the edible substrate 25, each containing their own different one or more APIs. Accordingly, by delivering multiple different API-containing liquids 31, a plurality of desired APIs can be delivered to the substrate 25. The API-containing liquids 31 can be delivered in the same quantity or in different quantities. Accordingly, the ratio of one or more APIs relative to one or more other APIs can be controlled. In other examples, the API-containing liquid 31 to be delivered to the substrate 25 can include a plurality of APIs, either in equal proportions or in desired ratios. Thus, a single API-containing liquid 31 can be delivered to the edible substrate 25 to deliver either a single API or a plurality of APIs. The plurality of API-containing liquids 31 delivered to the edible substrate 25 can include greater than one API up to the full range of APIs, which in the case of a cannabinoid can be approximately 113 different cannabinoids.

Because the API-containing liquid 31 is added to the food product after the food product has been prepared, it should be appreciated that the API in the API-containing liquid 31 can avoid exposure to elevated temperatures associated with a food preparation process. As a result, the API is not subject to processes that might otherwise degrade the efficacy of the API in the API-containing liquid 31. The present disclosure recognizes, however, that the methods of delivering the API to prepared food product can further be applied to raw food product, such as raw fruit and nuts, and other non-edible substrates.

According to an aspect, the API-containing liquid 31 can include one or more modifiers, such as flavonoids or the like, that are configured to modify at least one or more of flavor, mechanical properties, or aesthetics of the API in the API-containing liquid 31.

According to one example, energy can be added to the edible substrate 25 to increase adhesion of the API-containing material to the edible substrate 25 within the voids 32. For example, energy can be added to increase a temperature of the edible substrate 25. For instance, the temperature of at least one or both of the base 37 and the at least one internal surface 35 can be increased by applying conductive heat, convective, or radiation heat, examples of which are forced gas, microwaves, and light, such as infrared light. The energy can be added prior to, during, or immediately after delivering the API-containing liquid 31 into the voids 32, after delivering the API-containing material into the voids 32, or both before and after delivering the API-containing material into the voids 32.

All of the above method steps and apparatus described herein, including an active-containing substrate 25, can be incorporated into or provided by any suitable system such as the infusion dosing system 20. It will be appreciated that the system 20 can provide a cost-effective, efficient, and reliable method for providing a product line of substrates having desired amounts and types of APIs. While one such system 20 is illustrated and described herein, it is recognized that numerous alternatives are available for dispensing an approximate dose of the active pharmaceutical ingredient into the edible substrate 25 as described above.

When the at least one active pharmaceutical ingredient is to be delivered as an API-containing liquid 31, the system 20 can include at least one holding tank 42 that is configured to retain the API-containing liquid 31. The API-containing liquid 31 can be a solution of the type described above or a pure API for instance as an oil. The approximate concentration of the active pharmaceutical ingredient in the API containing material can be a known concentration described above. Thus, the API can define the solute of the solution, and the solution can define any suitable solvent. In one example, the solvent can be an alcohol, such as ethanol or any alternative alcohol as desired, or any other viscosity reducing agent as desired. In one example, the liquid 31 can contain a concentration of the active pharmaceutical ingredient that is in a range from approximately 40% to approximately 90%, such as from approximately 50% to approximately 70% by volume in solution with the solvent. It is recognized that the solvent can be removed substantially in its entirety during a subsequent drying step. For instance, the solvent can easily evaporate after being applied to the edible substrate 25. Nevertheless, it may be desired for the solvent to be safe for consumption in trace amounts.

Alternatively, the API-containing liquid 31 can be a stand-alone extract, meaning that it is not mixed with a carrier that is designed to be burned or otherwise evaporated off. The extract can be purified, partially purified, or unpurified as desired. It is recognized that such stand-alone extracts can be in the form of a resin having a relatively high viscosity that can prevent the extract from being suitably free flowing for easy delivery to the substrate 25. Therefore, as described in more detail below, the infusion dosing system 20 can include one or more heaters that are configured to raise the temperature of the extract, thereby lowering the viscosity of the API-containing liquid. Alternatively or additionally, an additive such as alcohol can be added to the liquid 31 that lowers the viscosity of the liquid. The alcohol readily evaporates after the liquid 31 has been applied to the substrate 25. It is recognized that the extract having a suitably low viscosity can be readily delivered to the substrate in any manner described herein. For instance, it can be desirable to maintain the extract at a heated temperature during application of the extract to the substrate 25. The heated temperature can range from about 0 degrees F. to about 200 degrees F., such as about 150 degrees F. to approximately 180 degrees F. Although it is envisioned that the extract has a sufficiently low viscosity at room temperature, it may nevertheless be desirable in some instances to maintain the solution at the heated temperature. Because the approximate dose of the at least one API in the API-containing liquid 31 is known, a predetermined approximate volume of the liquid 31 delivered from the holding tank 42 to the dosing head 34, and thus to the edible substrate 25, can contain approximately a predetermined approximate desired dose of the pharmaceutical ingredient.

Therefore, unless otherwise indicated, reference herein to API-containing liquid 31 disposed in the voids 32 can apply with equal force and effect to voids 32 that contain substantially only the API after the solvent has been evaporated or otherwise removed from the voids 32. Further, unless otherwise indicated, reference herein to API disposed in the voids 32 can apply with equal force and effect to voids 32 that contain API-containing liquid 31, for instance if the API-containing liquid was not evaporated or otherwise removed, or only partially evaporated or otherwise removed, from the voids 32 prior to closing the voids 32.

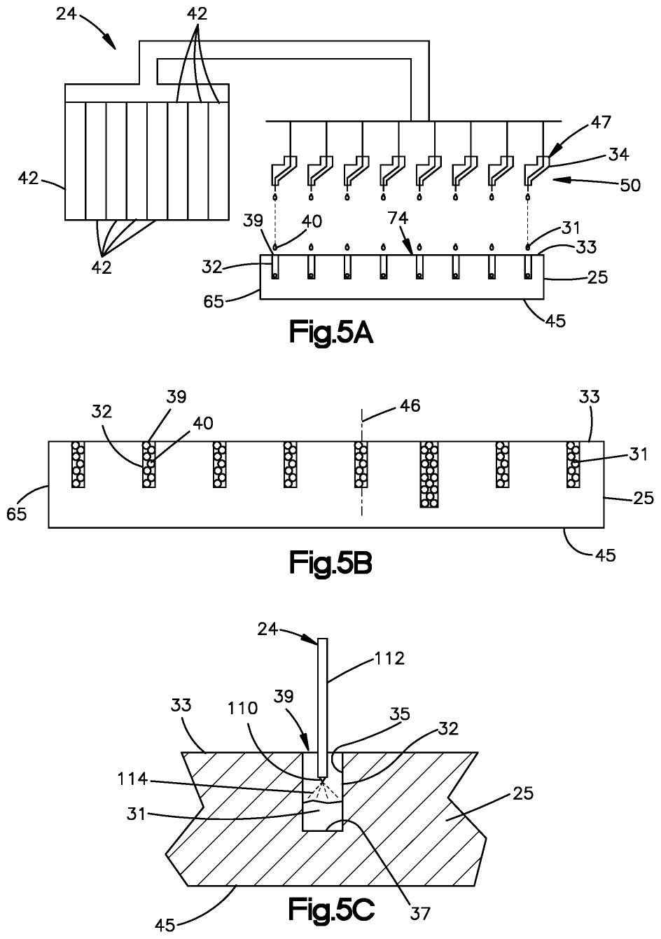

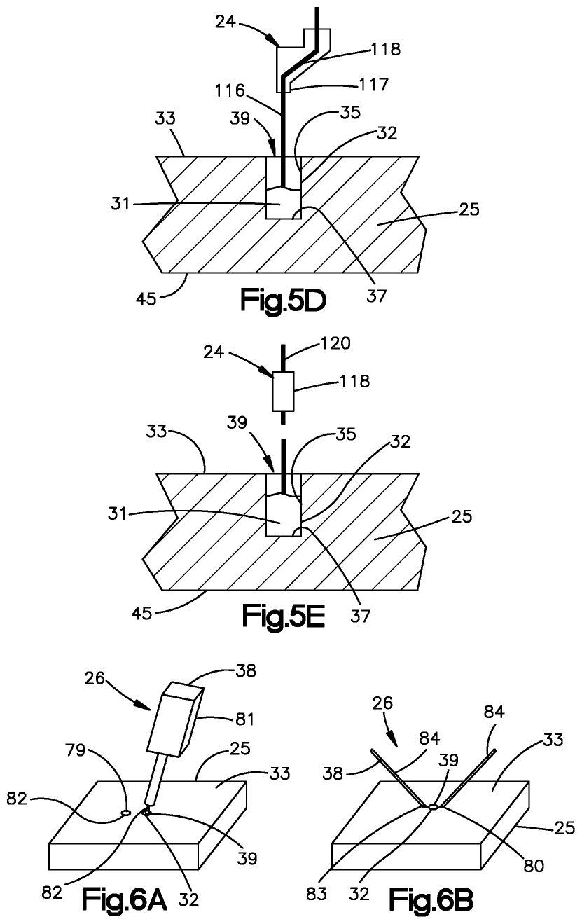

Referring now to FIGS. 5A-5B, the dosing station 24 will now be described in more detail. The dosing station 24 is configured to deliver at least one API-containing liquid 31 into at least one pre-formed void 32 to define a dosed void 32, and therefore a dosed edible substrate 25. The pre-formed void 32 can be an open void prior to delivery of the at least one API-containing liquid 31. The pre-formed void can be defined by the voids 32 that were created by the void forming station 22, or can be created in any suitable alternative manner as desired. Further, the pre-formed voids 32 can be created by the same entity that is performing the step of delivering the API-containing liquid 31 to the pre-formed voids 32. Alternatively, the pre-formed voids 32 can be created by a first entity that then delivers the edible substrates 25 having the voids to a second party who then performs the step of delivering API-containing liquid 31 to the pre-formed voids. The pre-formed voids 32 define an unfilled internal void prior to delivery of the API-containing liquid 31 into the pre-formed voids 32. The dosing station 24 can discontinue delivering the at least one API when a predetermined volume of the API-containing liquid 31 has been delivered from the dosing heads 34 into the respective voids 32. For instance, the dosing station 24 can discontinue delivering the at least one API-containing liquid 31 when a predetermined volume of API-containing liquid 31 has been delivered into the respective voids 32.

While the dosing station 24 will be described as including a plurality of dosing heads 34 configured to deliver an API-containing liquid 31 to a corresponding plurality of voids 32, it should be appreciated that the dosing station 24 can alternatively include only a single dosing head that can deliver an API-containing liquid 31 to a plurality of voids 32 as desired. The dosing station 24 can include at least one holding tank 42, a plurality of dosing heads 34, and at least one conduit that places the at least one holding tank 42 in fluid communication with the plurality of dosing heads 34. The at least one holding tank 42 can contain a respective at least one API-containing liquid 31. In one example, the at least one holding tank 42 can be configured as a single holding tank 42 or a plurality of holding tanks 42 that deliver the same API-containing liquid 31 to each of the dosing heads 34. In still another example, the at least one holding tank 42 can include a plurality of holding tanks 42, whereby at least one of the holding tanks 42 contains an API-containing liquid 31 that is different than the API-containing liquid disposed in at least one other of the plurality of holding tanks 42. Therefore, the dosing heads 34 can deliver various API-containing liquids 31 as desired so as to define a desired API profile across the edible substrate 25.

When the food product is positioned such that the pre-formed voids 32 are in alignment with the dosing heads 34, the API-containing liquid 31 can delivered from the dosing heads 34 to the respective aligned voids 32 of the plurality of voids 32. In one example, the voids 32 can be vertically aligned with the dosing heads 34 along the direction of gravity. While the dosing station 24 is described as configured to deliver microdroplets 40 from the dosing head 34, it should be appreciated that the dosing station 24 can alternatively be configured to deliver the API-containing liquid 31 in manner as described herein, for instance as drops, a spray, a stream, or a squirt as desired. As described herein, the microdroplets 40 can contain a predetermined approximate quantity of API. Thus, the dosing heads 34 can deliver a plurality of microdroplets 40 sequentially and individually into the aligned voids 32, thereby delivering a predetermined quantity of API based on the number of microdroplets 40 and the known quantity of API in each microdroplet. In particular, because the concentration of API in the liquid 31 is known, and the desired dose of the active pharmaceutical ingredient to be delivered to the void 32 is known, the approximate volume of the liquid 31 to be delivered to the void 32 can be determined. Based on the number of voids 32 that have received the API, the total quantity of API in the edible substrate 25 can be determined. The dosing station 24 can be configured to deliver the approximate volume of the liquid 31 to one void 32 at a time, or can be configured to deliver a plurality of approximate volumes of the liquid 31 to a respective plurality of the voids 32 simultaneously. The dosing station 24 can be configured as any suitable commercially available ultra-low volume liquid handling machine.

As described above, each dosing head 34 can be configured to dispense a respective approximate quantity of the approximate volume of the API-containing liquid 31 that is delivered from the holding tank 42. Thus, the dosing station 24 can include at least one dosing head 34 such as a plurality of dosing heads 34 that receive respective quantities of the API-containing liquid 31 delivered from the respective holding tank 42, and dispense the respective quantities into the voids 32. The respective quantities dispensed by the dosing heads 34 cumulatively define the approximate volume of API-containing liquid 31 that has been received by the voids 32.

As will now be described, the dosing heads 34 can be configured to deliver precise quantities of the volume of the API-containing liquid 31 to the voids 32. In some examples, the precise quantities can be microquantities defined by the microdroplets 40. Thus, the voids 32 of the edible substrates 25 can receive repeatable and predictable dosages of one or more APIs in the voids 32. Further, the dosage of the active pharmaceutical ingredient can be applied at specific locations of the edible substrate 25 as desired. For instance, in certain examples, it may be desirable to deliver an API into the voids 32 such that the API it is substantially evenly distributed on or in the edible substrate 25. As a result, for instance when the edible substrate is a large baked good, consumption of different regions of the edible substrate in equal volumes will cause ingestion of substantially identical quantities of the active pharmaceutical ingredient. In one example, the dosing heads 34 can be defined by a True Volume™ Piston Positive Displacement Pump commercially available from Creative Automation Company having a place of business in Sun Valley, CA. In another example, the dosing heads 34 can be defined by a Pipetman M P10M device commercially available from Gilson Inc., having a place of business in Middleton, WI.

In other examples, different dosing heads 34 can be configured to deliver different quantities of the respective volume of API-containing liquid 31 into the aligned voids 32. Further, the API-containing liquid 31 delivered by the first at least one dosing head 34 can include a different active pharmaceutical agent than the liquid 31 delivered as a second volume of API-containing liquid by a second at least one dosing head 34. Further still, the dosing system 24 can be configured to deliver any number of API-containing liquids 31 each containing a different pharmaceutical agent to a respective at least one dosing head 34. Accordingly, the dosing heads 34 can combine to deliver different APIs-containing liquids 31 into different ones of the voids 32. In one example, the different API-containing liquids 31 can contain the same API but in different concentrations. In other examples, the different API-containing liquids 31 can contain different APIs. The system 20 can therefore include any number of holding tanks 42 as desired, each tank one API in different concentrations. The different liquid extracts can be delivered to different respective ones of the dosing heads 34, for instance from respective tanks, and subsequently into different ones of the voids 32. Thus, different dosing heads can be configured to deliver different APIs to the edible substrate 25. For instance when the API is a cannabinoid, the different dosing heads can be configured to deliver different cannabinoids to the edible substrate 25.

As one example, a first group of dosing heads 34 can be configured to deliver a dose of a first active pharmaceutical agent, and a second group of dosing heads 34 can be configured to deliver a dose of a second active pharmaceutical agent, wherein the second active pharmaceutical agent is different than the first active pharmaceutical agent. For instance, the first active pharmaceutical agent can be THC, and the second active pharmaceutical agent can be CBD. It should be appreciated, however, that the first API can be a first any one or more of the APIs described herein, and the second API can be a second any one or more of the APIs described herein different than the first API. Further, the first API can be delivered in a different predetermined approximate dose than the second API. Further still, the tank containing the first API can be maintained at a different temperature than the second tank that contains the second API. Thus, the viscosity of each of the respective API-containing liquids 31 that contain the first and second APIs, respectively, can be individually controlled.

As described above, the dosing station 24 can be configured to deliver heat to the API-containing liquid 31 either prior to delivering the liquid 31 to the dosing heads 34, or after delivery to the dosing heads 34, which can be prior to or during dispensing of the API containing liquid 31 to the voids 32. The heat can be sufficient to decrease the viscosity of the API-containing liquid 31. Thus, in one example, the API-containing liquid 31 can include the API and solvent, and can travel from the holding tank 42 to the dosing head 34. The API-containing liquid 31 can be heated between the holding tank 42 and the dosing head 34 to decrease the viscosity of the liquid 31. Alternatively or additionally, the API-containing liquid 31 can be heated at the dosing head 34 so as to decrease the viscosity of the liquid 31. Alternatively, in some examples such as when the liquid 31 is a solution, the liquid 31 can be maintained at room temperature.

The dosing heads 34 can be arranged in an array 47 that includes at least one row 50 of dosing heads 34. The dosing heads 34 of each row 50 can be substantially equidistantly spaced along the respective row 50. Alternatively, the dosing heads 34 can be variably spaced along the respective row 50. The array 47 can further include a plurality of columns that space the rows 50 from each other. The dosing heads 34 can be equidistantly spaced along the respective columns 52. Alternatively, the dosing heads 34 can be variably spaced along the respective columns 52. In one example, all of the dosing heads 34 can be configured to deliver the same at least one active pharmaceutical ingredient. Alternatively, as described above, different groups of the dosing heads 34 can be configured to deliver a respective different active pharmaceutical ingredients. Each group can include at least one dosing head 34 up to a plurality of the dosing heads 34. Each group can be defined by a respective one or more of the rows 50. Alternatively, each group can be defined by a respective one of the columns.

Referring now to FIGS. 1A and 5A-5B, groups of one or more of the dosing heads 34 can be aligned with different respective locations of a dosing zone 74 (see also FIG. 3C) of the edible substrate 25. That is, one or more groups of the recesses 32 can be disposed in one or more dosing zones 74. Accordingly, the dosing heads 34 can be positioned to deliver their respective quantities of the volume of liquid 31 to the different dosing zones 74. Further, the system 20 can be configured to deactivate select dosing heads 34 that are out of alignment with respective voids 32, and activate select dosing heads 34 that are aligned with respective voids and thus receive respective the API-containing liquid 31. The dosing zones 74 can be disposed inside the outer perimeter 65 of the edible substrate 25. For instance, the dosing zones 74 can be dispose in an area that is greater than half, for instance greater than 75%, of a footprint defined by the outer perimeter. Either way, it can be said that each dosing zone 74 can be disposed at predetermined location with respect to each other and the outer perimeter 65 of the edible substrate 25. Thus, the dosing zones 74 can be consistent among a plurality of differently sized edible substrates 25, such as cookies or brownies that can have similar but non-identical sizes and shapes.

The dosing heads 34 can be spaced from each other as desired so as to deliver a desired distribution of the active pharmaceutical ingredient to the edible substrate 25 in the voids 32. For instance, the dosing heads 34 can be arranged in a plurality of rows and columns that correspond to the rows and columns of the openers 30, and thus the rows and columns of the voids 32. In one example, the dosing heads 34 of the same group can be configured to deliver substantially the same volume of API-liquid 31 to the edible substrate 25 in the respective dosing zone 74. Further, the dosing heads 34 of the same group are configured to deliver the same API-liquid 31 to the edible substrate 25 the respective dosing zone 74. Different groups of dosing heads 34 can be configured to deliver one or more of different quantities and different constituent components to the voids 32 of the respective dosing zones 74.

While the dosing heads 34 can be stationary at some point with respect to movement in a direction angularly offset to the central axes of the aligned voids 32 in some examples, in other examples the dosing heads 34 can be movable in the direction so as to deliver the API-containing liquid 31 to multiple voids 32. Thus, at least one dosing head 34 such as a plurality of dosing heads 34 can be movable along the substrate 25 so as to deliver the respective at least one active pharmaceutical ingredient at into different voids 32 of the edible substrate 25. The different voids 32 can belong to the same group or can be long to different groups 54.

In still other examples, one or more of the dosing heads 34 can be configured to deliver different combinations of API-containing liquids. For instance, a dosing head 34 can deliver to a select void 32 a first API-containing liquid, and can subsequently deliver to the select void 32 a second API-containing liquid different than the first API-containing liquid. Next, the dosing head 34 can deliver to the select void a third API-containing liquid that is different from each of the first and second active pharmaceutical ingredients, and so forth until all desired API-containing liquids have been delivered to the select void 32.

Thus, numerous examples are presented whereby the dosing heads 34 are configured to deliver one or more APIs to different locations of the edible substrate 25. The dosing heads 34 can remain stationary with respect to the edible substrate 25 as the respective API-containing liquid is delivered to the substrate 25. Alternatively, the dosing heads 34 can be movable along the edible substrate 25, such that the various API-containing liquids delivered by different dosing heads 34 can be delivered to the same respective location of the substrate 20. The same or different API-containing liquids at the different respective locations can be distributed as desired in at least one or more directions along the edible substrate 25.

Continuing with reference to FIG. 5A, the dosing heads 34 can be disposed external of the edible substrate, and spaced from the edible substrate 25 along a direction of travel of the microdroplets 40 from the dosing heads 34 to the voids 32. Thus, the active pharmaceutical ingredient is delivered to the substrate along the direction of travel. In one example, the dosing heads 34 are spaced above the edible substrates 25 along a vertical direction, such that the direction of travel is aligned with gravitational forces. The direction of travel can be coincident with or otherwise parallel to the central axis 46 of the void 32. The dosing heads 34 can be spaced from the edible substrates 25 any suitable distance when delivering the active pharmaceutical ingredient to the edible substrates 25, such as from approximately 2 mm to approximately 25 mm. As shown at FIG. 5A, at least some of the microdroplets 40 up to all of the microdroplets 40 can be substantially spherical shaped. Alternatively or additionally, at least some of the microdroplets 40 up to all of the microdroplets 40 can be elongated, such as substantially teardrop shaped or alternatively shaped as desired. For instance, surface tension of the microdroplets 40 with the dosing heads can cause the microdroplets 40 to be teardrop shaped as they leave the dosing heads, and can transition to substantially spherically shaped as they travel to and into the voids 32.

In one example, the microdroplets 40 are delivered from the dosing heads 34 to the respective locations of the edible substrate 25 under any suitable force, such as gravitational forces, electrostatic forces, or the like. In another example, the microdroplets 40 are delivered from the dosing heads 34 to the respective locations of the edible substrate 25 under positive pressure. In still other examples, one or more of the dosing heads 34 can be coupled to respective needles that can be inserted into the pre-formed voids 32 so as to deliver the respective microdroplets 40 through the needles and into the voids 32 in the manner described above. In some instances, the needle can be heated at a temperature suitable to control the viscosity of the API-containing liquid 31 as it exits the needle. In one example, the needles can have a cross-sectional dimension sized less than that of the voids 32 such that the needles do not enlarge the voids 32 as they are inserted into the voids 32. In other examples, the needles can be sized to enlarge the voids 32 as desired.

As described above, the edible substrate 25 can be configured as any suitable food product as desired. In some examples, food product can have relatively low surface areas and correspondingly low volumes. Examples of such food product can include small or bite-size candies, nuts, and raw or dried fruits. It is recognized that variations in the dosage of API applied to such food product can have a greater impact on the ratio of API per volume of edible substrate when compared to food products having larger surface areas and volumes. Therefore, it can be particularly advantageous to precisely control the dosage of active pharmaceutical ingredient added to such food products. The APIs applied to the food products as microquantities, for instance defined by microdroplets 40, can allow for the precise control of the dosage of active pharmaceutical ingredients applied to the food products. Depending on the size of the food products, it is recognized that microdroplets having respective volumes ranging from approximately 5 nanoliters to approximately 20 microliters can be delivered into the voids 32. Thus, each food product can include a quantity or dosage of API in the range from approximately 2.5 micrograms to approximately 100 milligrams. Accordingly, each microdroplet can contain a microquantity of API in a range from approximately 0.5 micrograms to approximately 1 milligram. It is recognized, of course, that the dosage of API per food product can vary as desired. For instance, other quantities of microdroplets 40 can be delivered to food products, for instance depending on the size of the food product and corresponding number and size of voids 32, the size of the microdroplets 40, and the concentration of API in the microdroplets 40. The microquantity of API in a microdroplet 40 can allow the dosage of API delivered to a substrate to be precisely controlled as described above based on the number of microdroplets delivered into the voids 32. Further, the dosage of API per food product can be precisely controlled, as can a plurality of food products that amount to a single serving of the food products. It is appreciated that for larger edible products, such as baked goods, the microdroplets 40 can be applied in the range of approximately 5 nanoliters to approximately 20 microliters over the entire food product, and can greatly increase the total API delivered to the food product to numerically large amounts as desired. Therefore, microquantities of the active pharmaceutical ingredients can be repeatably delivered into each edible substrate 25, thereby producing consistent desired dosing across a plurality of edible substrates 25. Thus, the dosage of the at least one active pharmaceutical ingredient carried by the substrate 25 can be better controlled with respect to conventional application processes. It should be appreciated, however, as will now be described, that the API-containing liquid 31 can be delivered to the voids 32 in any suitable manner as desired.

While the API-containing liquid 31 can be delivered in the form of microdroplets 40 in the manner described above with respect to FIGS. 4A-5B, the API-containing liquid 31 can alternatively be delivered in any suitable alternative form as desired. For instance, the API-containing liquid 31 can be delivered as drops of any size as desired. Thus, the dosing head 34 of FIG. 4A can be configured to deliver drops having a larger size than the microdroplets 40 described above.

In another example, referring now to FIG. 5C, the dosing station 24 can be configured to deliver the API-containing liquid 31 as a spray 114. In particular, the dosing station 24 can include a dosing head configured as a spray nozzle 110 and a conduit 112 that delivers the API-containing liquid 31 to the spray nozzle 110 which can deliver a spray 114 into the void 32. In one example, the spray nozzle 110 can be sized to fit into the void 32. It is appreciated that the void 32 is fully created prior to insertion of the spray nozzle 110 into the void 32. Thus, the spray nozzle 110 does not create the void 32. In other examples, the spray nozzle 110 can be located external of the void 32 and can direct the spray 114 into the void 32. The delivery of the spray 114 can be discontinued once a predetermined volume of the API-containing liquid 31 has been delivered into the void 32.

In still another example, referring now to FIG. 5D, the dosing station 24 can be configured to deliver the API-containing liquid 31 as a stream 116. In particular, the dosing station 24 can include a dosing head configured as a dispensing nozzle 117 and a conduit 118 that delivers the API-containing liquid to the dispensing nozzle 117. The dispensing nozzle 117 can deliver the stream 116 of API-containing liquid 31 into the void 32. The dispensing nozzle 117 can be located exterior of the void 32, and can direct the stream 116 into the opening 39, for instance substantially along the central axis 46 of the void 32. Alternatively, the dispensing nozzle 117 can be inserted into the opening 39 and deliver the stream 116 while inside the void 32. The stream 116 can be discontinued once it has been determined that a predetermined volume of the API-containing liquid 31 has been delivered into the void 32. The stream 116 can be a continuous stream or a segmented stream as desired.

In yet another example, referring now to FIG. 5E, the dosing station 24 can be configured to squirt the API-containing liquid 31 into the void 32. The dosing station 24 can be configured as a manual squirter having a cylinder 118 and a plunger 120 that is configured to be depressed during a stroke so as to cause a predetermined volume of API-containing liquid 31 to be delivered from the cylinder 118 out of a dosing head and into the void 32. In other examples, the dosing station 24 can be configured as an automatic squirter. The dosing station 24 can be configured to deliver at least one or more squirts of the API-containing liquid 31 into the void 32 until a desired volume of the API-containing liquid 31 has been delivered. It should be appreciated that the concentration of API in the API-containing liquid 31 can be known, and thus the dosage of API delivered into the void 32 is known based on the volume of API-containing liquid 31 that is delivered into the void 32.

While certain examples of dosing stations 24 have been shown and described, it is recognized that any suitable dosing station configured to deliver the API-containing liquid 31 or an API powder into a pre-formed void 32 is contemplated. It is further recognized that in all instances, the edible substrate 25 is disposed such that the void 32 is in operable alignment with the dosing head. Accordingly, the dosing head, whether configured to deliver microdroplets, drops, a spray, a stream, a squirt, or any other form, is configured to deliver the liquid-containing API into the void.

If desired, the infusion dosing system 20 can include at least one drying head or a plurality of drying heads that are configured to deliver a drying agent to the edible substrate 25 as a whole, or to select groups of one or more up to all of the voids 32 so as to dry the API-containing liquid 31 in the voids 32. In other examples, the API-containing liquid 31 in the voids can remain undried. It is appreciated that when the liquid 31 dries, it does so by evaporating the solvent, leaving the active pharmaceutical ingredient in the substrate 25.

The liquid 31 can be dried by applying conductive heat, convective, or radiation heat, examples of which are forced gas, microwaves, and light, such as infrared light. The forced gas can be delivered to the outer surface 33 of the edible substrate 25. The forced gas can be air, nitrogen, or any suitable alternative gas such as an inert gas. The forced gas can be heated, and can have a temperature that is in a range for instance from about 100 degrees F. to about 250 degrees F. Alternatively, the forced gas can be substantially unheated, and thus at ambient temperature. Alternatively still, the forced gas can be cooled, and thus at a temperature below ambient temperature. In this regard, the cooled forced gas can cause an API to-reduce or eliminate their evaporation of the solvent so as to allow the API-containing solution to impregnate the thickness of the substrate 25 if desired. Alternatively, the post-processing station 40 can expose the dosed edible substrate 25 to ambient air or a controlled environment so as to dry the volume of liquid 31.

It should be appreciated that several advantages can be achieved using the dosing station 24. In one example, because the API is disposed in the edible substrate 25, the initial flavor profile of the edible substrate 25 upon placement in the mouth is defined by the constituent components of the edible substrate 25, and not of the API. In other examples, of course, it should be appreciated that the API-containing liquid 31 can be delivered to an outer surface of the substrate 25 as well, for instance if it is desired to impart a flavor profile from the API onto the substrate surface. Further, the edible substrate 25 can include multiple active pharmaceutical ingredients, which can eliminate a conventional need to consume multiple medications each having a single active pharmaceutical ingredient. Additionally, the at least one active pharmaceutical ingredients can be distributed in a desired profile (e.g., substantially evenly or variably) along the edible substrate 25. Further still, individual dosing of the active pharmaceutical ingredients on the substrate can allow for the use of locally produced active pharmaceutical ingredients that are applied after the substrate has been prepared, thereby avoiding the need to transport the applied active pharmaceutical ingredients over long distances with varying environmental control or across jurisdictional boundaries.

It should be further appreciated that the dosing station 24 can be used to dose a plurality of substrates 25 that provide a dosing regimen over a period of time. For instance, the dosing station 24 can dose a plurality of substates 25 consistently in the manner described above. Alternatively, the dosing station 24 can dose a plurality of substrates 25 having different API characteristics from each other. The dosing station 24 can deliver respective APIs into the voids 32 of each of the substrates, where the respective APIs of each substrate 25 have at least one API characteristic that is different than at least one other substrate 25. For instance, a first API-containing liquid or plurality of API-containing liquids can be delivered to a first at least one substrate 25, such as a first plurality of substrates 25. A second API-containing liquid or plurality of API-containing liquids can be delivered to a second at least one substrate 25, such as a second plurality of substrates 25. The first and second APIs can have at least one API characteristic that is different from each other. The different API characteristic can include at least one of 1) a concentration of the API, 2) a volume of API delivered to the substrates during the delivering step, 3) a composition of the API, 4) a modifier mixed with the API, the modifier configured to modify at least one of a flavor, a mechanical property, and an aesthetics of the delivered API, and 5) a location of at least one dosing zone of the substrate 25 that defines a location of the substrate 25 where the API-containing liquid is to be deposited. The mechanical property can include viscosity of the API-containing liquid in some examples. The mechanical property can further include the surface tension of the API-containing liquid that is delivered from the dosing station. It is further appreciated that the different API characteristic can include a different predetermined dosage that is delivered to the different substrates 25. In one example, the dosage can be predetermined to correspond to a dosing regimen over a period of time. Thus, groups of one or more substrates can have dosages that differ and are designed to be consumed at predetermined times over the course of the dosing regimen. For instance, the dosage can decrease over the period of time defined by the dosing regimen. Alternatively, each substrate 25 can receive API-containing liquid 31 having the same API characteristics. Further, different groups of substrates can receive API-containing liquid 31 with at least one different API characteristic, wherein all substrates among each group receive the same API-containing liquid 31.

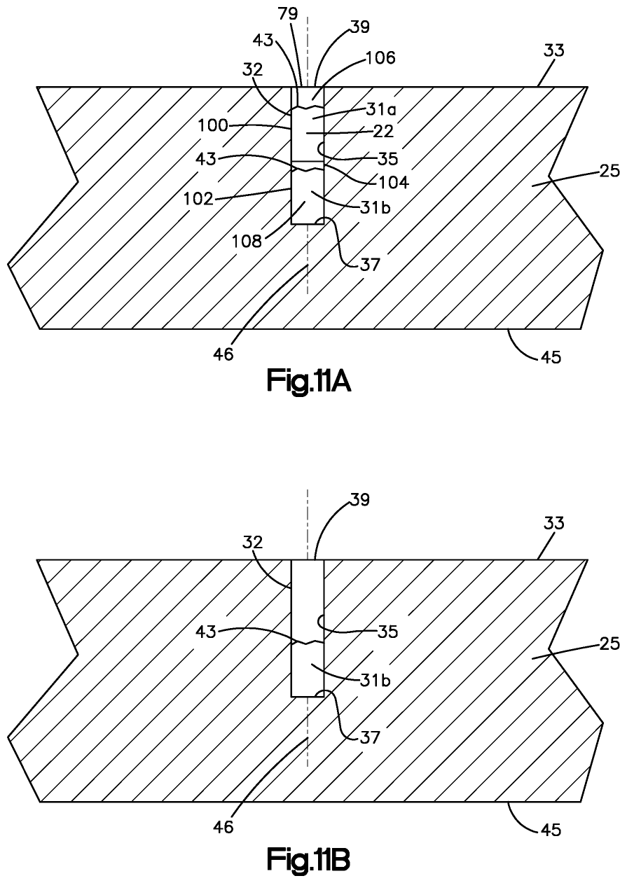

In some examples, the edible substrates 25 can be substantially non-porous, such that the API-containing liquid 31 delivered into the voids 32 remains in the voids 32. In other examples, the edible substrates 25 can be porous, such that the API-containing liquid 31 can travel across either or both of the internal surface and/or the base 37 and into the pores of the edible substrate 25. Referring now to FIGS. 10A-10B, in still other examples whereby the edible substrate 25 is porous, either or both of the internal surface 35 and the base 37 can be treated so as to define a non-porous barrier 76 at the internal surface 35 and/or the base 37. The non-porous barrier 76 can be impermeable with respect to the API disposed in the respective void 32, such that the API is unable to travel across the barrier 76 into the pores of the edible substrate 25. Thus, when the barrier 76 is applied to an entirety of each internal surface 35 and an entirety of the base 37, the void 32 in combination with the barrier 76 can define a silo that causes all of the API in the void 32 to remain in the void 32 prior to consumption of the edible substrate 25. As will be described in more detail below, the API can be ingested transmucosally, can be ingested via intestinal absorption, or a combination of both.

For instance, as shown in FIGS. 10A-10B, a delivery device 71 can be inserted into the void 32 and apply a spray 73 onto either or both of the internal surface 35 and/or the base 37 so as to define a non-porous barrier 76 along at portion up to an entirety of the internal surface 35. The spray can alternatively or additionally define a non-porous barrier 76 along a portion up to an entirety of the base 37. When a non-porous barrier 76 is defined along an entirety of the base 73 and an entirety of the internal surface 35, the void 32 can define a silo that prevents migration of the API-containing liquid through both the internal surface 35 and the base 37 to an adjacent region of the edible substrate 25.

The spray 73 can be non-porous with respect to migration of the API-containing liquid out of the void and into an adjacent region of the edible substrate 25. The spray 73 can be applied as the delivery device 71 travels along the central axis 46 of the void 32 inside the void 32 so as to coat the base 37 and the internal surface 35. In other examples, the delivery device 71 can be located outside the void 32 and direct the spray 73 into the void 32. In some examples, the spray 73 can be a spray of liquid barrier material. The liquid spray 73 can dry and harden, thereby creating a solid shell that, in turn, defines the non-porous barrier 76. The solid shell can be formed along the internal surface and/or the base 37. For instance, the solid shell can be formed on the internal surface 35 and/or the base 37. Alternatively or additionally, the solid shell can be formed slightly in the edible substrate 25 and along the internal surface and the base 37. In other examples, the liquid spray 73 can be applied as microdroplets of liquid barrier material in the manner described above. The microdroplets of liquid barrier material can wet the internal surface 35 and/or the base 37, and can dry and harden, thereby creating the solid outer shell. In still other examples, the liquid API can be delivered as microdroplets into the void after the liquid barrier material has been deposited into the void 32. In other examples, the liquid API can be mixed into the liquid barrier material prior to delivering the liquid barrier material is delivered to the internal surface and/or base 37. Thus, delivery of the liquid barrier material into the void 32 can also deliver the API into the void 32. The liquid barrier material can solidify along the internal surface 35 and/or base 37, thereby leaving the liquid API in the void 32. The liquid API can be delivered to the liquid barrier material in any suitable manner as desired, including as microdroplets.

In other examples, the spray 73 can be a solid spray. Solid particulates of the solid spray can define a coating having a sufficient depth that creates the non-porous barrier 76. In some examples, the solid particulates can be heated and subsequently cooled so as to create a solid non-porous shell that, in turn, defines the non-porous barrier 76. In some examples, the solid particulates are liquified when heated, and subsequently solidified. In one embodiment the solid spray 73 can be sugar coats the internal surface 35 and the base 37. Cooling of the sugar can then define the non-porous barrier 76. In one example, steam can flow into the void 32 and cause the solid, such as sugar, to precipitate and subsequently solidify during cooling. The steam can flow into the void 32 without inserting a steam delivery member into the void 32. Rather, the edible substrate 25 can be placed in a suitable environment such that the steam has sufficient transport to enter the void 32. The non-porous barrier 76 can be made from any suitable edible material, such as one or more edible adhesives such as binders. In some examples, the internal surface 35 and base 37 can be coated with an adhesion layer prior to applying the spray 73.

While the non-porous barrier 76 can be defined by a spray in some examples, in other examples a barrier material can be deposited into either or both of the internal surface 35 and the base 37 using, for instance, chemical vapor deposition (CVD) or physical vapor deposition (PVD). In the case of CVD, the barrier material can be applied to the internal surface 35 and the base 37 without inserting the delivery device 71 into the void 32. Rather, the edible substrate 25 including all voids 32, or a select one or more of the voids 32, can be placed into any suitable environment such that the chemical vapor or gas has sufficient transport to enter the void 32 and deliver the solid barrier material onto the internal surface 35 and the base 37. Either or both of the internal surface 35 and the base 37 can be pretreated as desired to allow the solid barrier material to be applied only in predetermined regions in either or both of the internal surface 35 and base 37.

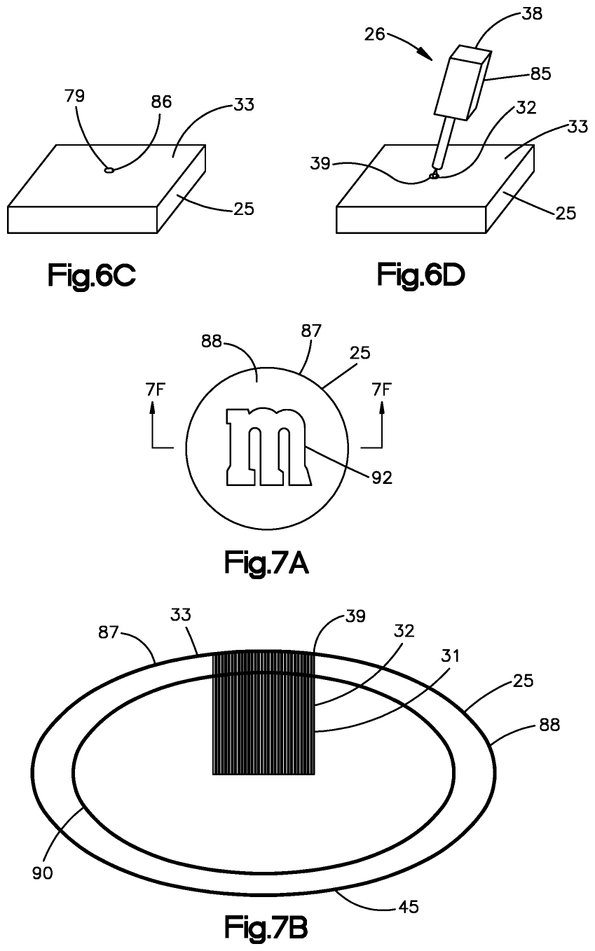

Referring now to FIGS. 6A-6C, the closing station 26 can include at least one such as a plurality of closing devices 38 that are configured to close the opening 39 at the first or outer surface 33 of the edible substrate 25, thereby closing the void 32 that defines the opening 39. As a result, the API disposed in the void 32 is prevented from flowing out of the void 32 and out of the edible substrate 25. It is recognized that the closing device 38 can be used to close the 39 particularly in instances whereby the edible substrate 25 is non-porous. In instances whereby the internal surface 35 and/or base 37 is porous, the opening 39 can remain open as desired, as the API can flow through the inner surface 35 and/or base 37 in the manner described above with respect to FIGS. 5A-5E. In other instance, even when the inner surface 35 and/or base 37 is porous, it may be desirable to close the opening 39.

It should be appreciated that after the drying step that dries the solvent, the voids 32 of the edible substrate 25 can contain the respective APIs in the voids 32. It should be further appreciated that the drying step can be optional, such that the voids of the edible substrate 25 contain API-containing liquids. Therefore, description of an API-containing liquid disposed in the void 32 applies with equal force and effect to an API disposed in the void 32, either with or without a liquid. Conversely, description of an API disposed in the void 32 applies with equal force and effect to an API-containing liquid disposed in the void 32.

The closing device 38 can be configured in any suitable manner desired. In one example shown at FIG. 6A, the closing device 38 can be configured as a delivery member 81 that is configured to deliver an edible material 82 to the first or outer surface 33, such that the edible material 82 covers the void 32 and thus closes the opening 39. In this regard, the edible material 82 can be referred to as a closure member 79 that closes the opening 39. The edible material 82 can be sized to cover the outer surface 33 without traveling into the void 32. In other examples, the edible material 82 can travel into the void 32, but a sufficient quantity of the edible material 82 can be delivered to the edible substrate 25 such that the edible material 82 travels into the void 32 at a sufficient depth in the void so that the edible material 82 extends to the outer surface 33. Thus, the edible material 82 can be disposed on the outer surface 33 adjacent the void, and can further extend across the void 32 so as to close the opening 39. The edible material 82 can extend continuously across a region of the outer surface 33 that defines a plurality up to all of the voids 32, thereby closing the openings 39 of the voids 32 in the region. Alternatively, the edible material 82 can be applied to the voids 32 individually or in groups of voids 32. The edible material 82 can, for instance, be configured as sugar, cooked or raw flour, ink, breadcrumbs, cornmeal, caramel, toffee, or any suitable alternative material as desired. In this regard, the edible material can be a solid material or a viscous material. The edible material 82 can be flavored as desired, for instance to match the flavor profile at the outer surface 33 of the edible substrate 25.

If desired, referring now to FIGS. 6A and 6D, the closing station 26 can include a heater 85 that is configured to deliver heat to the edible substrate 25, and in particular to the first or outer surface 33 of the edible substrate 25 adjacent the opening 39 of the void 32. In instances whereby the edible substrate 25 is configured to melt, soften, or sweat in response to heat, the application of heat can bond the outer surface 33 of the edible substrate 25 to the edible material 82. The edible material 82 can be configured as granules, flakes, particulates, one or more fruits, nuts, or can be provided in any suitable form as desired. In instances whereby the edible material 82 is configured to melt, soften, or sweat in response to heat, the application of heat can bond the edible material 82 to the outer surface 33. Either way, it can be said that the outer surface 33 and the edible material 82 can be bonded to each other. The outer surface 33 and the edible material 82 can be bonded to each other in any suitable alternative manner as desired. For instance, an edible adhesive can be applied to either or both of the outer surface 33 and the edible material 82. Thus, when the edible material 82 is applied to the outer surface 33, the edible material adheres to the outer surface 33 and covers the opening 39. In this regard, each edible material 82 can be dimensioned greater than the maximum cross-sectional dimension of the opening 39 along a direction perpendicular to the central axis 46 of the void 32. Accordingly, the edible material 82 can adhere to the outer surface 33 without traveling into the void 32.

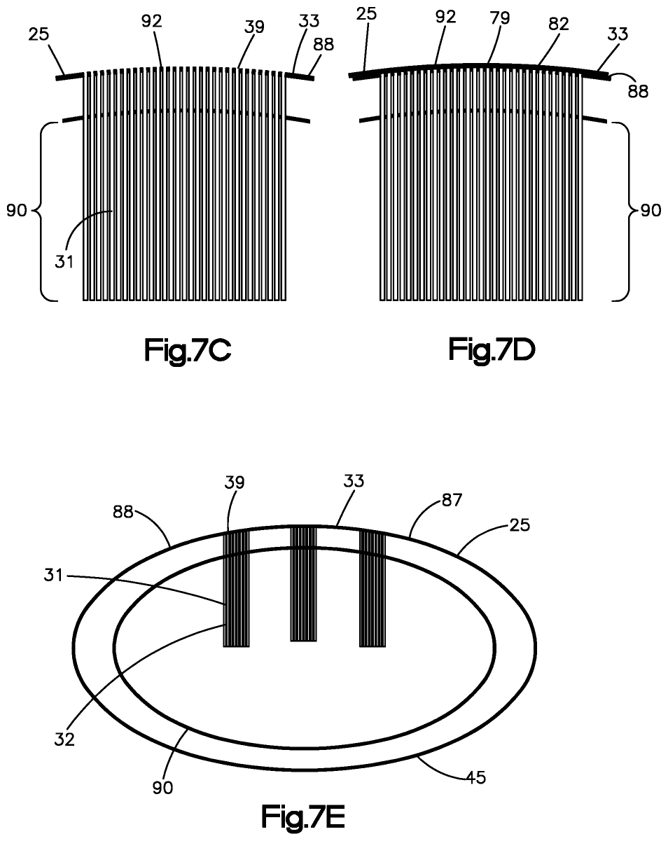

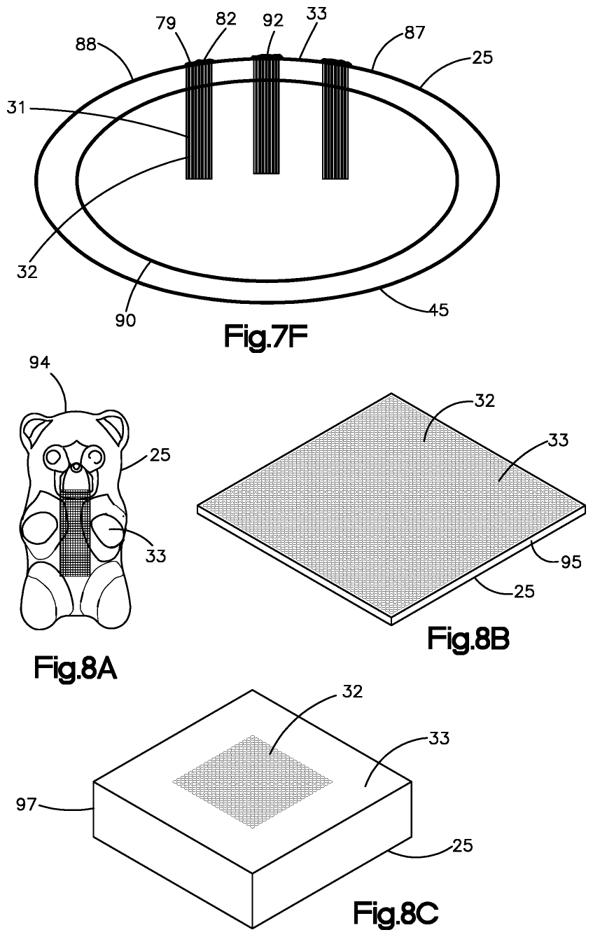

One such example is shown in FIGS. 7A-7F which shows the edible substrate 25 as a food product configured as an M&M® candy 87 having a hard outer shell 88 that surrounds a mass 90 of chocolate. The outer shell 88 can be initially provided as a liquid candy that surrounds the mass 90 of chocolate, and is subsequently solidified so as to define a hard candy outer shell 88. The outer shell 88 defines the outer surface 33. The voids 32 can extend through the outer shell 88 and into the mass 90 of chocolate. As shown in FIGS. 7C and 7E, the voids 32 can receive the API-containing liquid 31, and can define the openings 39 in the manner described above. As illustrated in FIGS. 7D and 7F, the edible material 82 can be applied to the outer surface 33 so as to close the openings 39.

In one example, the candy 87 can further have identifying indicia 92 defined by an edible material 82 carried by its outer surface 33. The indicia 92 can be defined by an ink or any suitable edible material as desired. In one example, at least some up to all of the voids 32 can be disposed in a region that is limited to those regions of the outer surface 33 that carry the indicia 92. The edible material 82 delivered to the outer surface 33 to close the openings 39 can therefore be defined by the same ink that defines the indicia 92. Alternatively or additionally, at least some up to all of the voids 32 can be disposed in a region that of the outer surface 33 that does not carry the insignia. In such examples, the edible material delivered to the outer surface 33 to close the openings 39 can be a liquid candy, and in particular the same liquid candy that defines the outer shell 88. The liquid candy can have a surface tension that limits or prevents the liquid candy from flowing into the voids 32. The liquid candy can subsequently harden such that the edible material 82 matches the outer shell 88 in both material and color. While the edible substrate 25 can be configured as an M&M® candy 87 as one example of a food product whose openings 39 can be closed with an edible material 82, it should be appreciated that the edible substrate 25 can define any suitable food product as desired.

Referring now to FIGS. 6B-6C, the closing device 38 can alternatively be configured to deform a region 80 of the edible substrate 25 so as to close the void 32. In this example, the edible substrate 25 can be malleable and plastically deformable at room temperature. Alternatively, the edible substrate can be malleable and plastically deformable at an increased temperature. In particular, the closing device 38 can be configured to physically displace a portion of the edible substrate 25 that includes the outer surface 33. For instance, the closing device 38 can be configured as one or more pusher members 84 that can be brought into contact with the first or outer surface 33 at a location adjacent the opening 39 void 32. The pusher member 84 can be driven to move in a direction toward the opening 39, which drives the region 80 of the edible substate 25 that includes the outer surface 33 to become displaced across the opening 39. In one example, a single pusher member 84 can drive the region 80 entirely across the opening 39 in one or more strokes of movement of the pusher member 84. The edible substrate 25 can be sufficiently tacky so as to bond with itself. Thus, the displaced region 80 of the outer surface 33 can travel across the opening 39 and bond with another adjacent region 83 of the outer surface 33. The closure member 79 can thus be configured as a bond of different regions of the edible substrate 25 to each other. The adjacent region 83 can be defined by the outer surface 33 that is on different, such as opposite, sides of the opening 39 with respect to the region 80 prior to displacing the region 80. In another example, the adjacent region 83 can be defined by a portion of the internal surface 35 that partially defines the void 32. When the region 80 is displaced, the region 80 and the adjacent region 83 can bond with each other, thereby defining a closed seam 86 that closes the opening 39, and thus closes the void 32. Accordingly, the closure member 80 can be configured as a closed seam. In other examples, the closing device 38 can include more than one pusher member 84, such as first and second pusher members 84 that can be configured to drive respective the regions 80 and 83 to each other and/or toward another adjacent region that is disposed adjacent the opening 39, so as to close the opening 39. The regions 80 and 83, and the other adjacent region if present can define respective portions of the interior wall 35 of the void 32. The edible substrate 25 can define any number of regions as desired, at least some of which can be displaced by at least one pusher member 84 so as to close the opening 39 in the manner described above.

Referring also to FIG. 8A, in one example the edible substrate 25 is shown as a food product configured as a gummy candy 94. In another example shown at FIG. 8B, the edible substrate 25 is shown as a food product configured as a fruit roll-up 95. In still another example shown at FIG. 8C, the edible substrate 25 is shown as a starburst® candy 97. Thus, the edible substrate 25 can be malleable and plastically deformable in the manner described above. Further, the edible substrate of FIGS. 8A-8B can be configured to melt, soften, and sweat, or otherwise become tacky in response to heat. Thus, the voids 32 can be formed in the manner described above, and the API-containing liquid can be delivered into the voids 32 in the manner described herein. Finally, the voids 32 can be closed in any manner desired. For instance, at least one pusher member can cause a corresponding at least one the region of the edible substate 25 to become displaced and bond to at least one other region of the substrate 25 across the opening 39 so as to close the opening 39 at a seam 86. Alternatively or additionally, an edible material can be applied to the outer surface 33 and over the opening 39 in the manner described above. The edible material can be the same edible material that defines the edible substrate 25 in some examples. While the edible substrate 25 can be configured as two examples of a food product shown in FIGS. 8A-8B whose openings 39 can be closed with at least one pusher member, it should be appreciated that the edible substrate 25 can define any suitable food product as desired.

Referring now to FIGS. 6B-6D, the closing station 26 can include the heater 85 that is configured to deliver heat to the edible substrate 25 in combination with the at least one pusher member 84. The heater 85 can be configured to deliver heat to the first or outer surface 33 of the edible substrate 25 adjacent the opening 39 of the void 32. In instances whereby the edible substrate 25 is configured to melt, soften, or sweat, or otherwise become tacky in response to heat, the application of heat can cause the regions 80 and 83 of the edible substrate 25 to bond with each other or with another adjacent region as described above, or can enhance the bonding. In some examples, the application of heat can cause the solvent of the API-containing liquid 31 to dry in the manner described above, and can also cause the opening 39 to close, alone or in combination with another closing device, such as the at least one pusher 84 or the delivery member 81.

In this regard, it should be appreciated in some examples that the application of heat alone can cause the outer surface 33 to melt and deform, thereby closing the voids 32. For instance, when heat is applied to certain edible substrates 25, and in particular food products that respond to heat by melting, softening, or sweating such as chocolate, baked goods, gummy candy, lollipop, and the like, the temperature of the surface of the edible product be raised to a level whereby the edible product melts, sweats, or otherwise assumes a form that is configured to encapsulate the API in the voids 32. The heater 85 can be configured to increase the temperature of at least one or more regions of the edible substrate 25, for instance, by directing at least one or more of heated forced gas and a light to the outer surface 33.

While the edible substrates 25 can be configured as food product in one example, and such food product can have advantages over dosage forms for reasons stated above, it may be desirable to alternatively deliver the APIs to a dosage form 96. Thus, in another example shown at FIG. 9, the edible substrates 25 can be configured as a dosage form 96, such as a tablet, pill, or any suitable alternative dosage form as desired. Because multiple APIs can be delivered to the voids 32 of the dosage form 96, fewer dosage forms are required to be ingested in order for the desired APIs to be delivered to the consumer with respect to conventional dosage forms. For instance, where it may have been necessary to consume several dosage forms during a given day in order to ingest a specific plurality of APIs, the consumer can now consume fewer dosage forms, such as a single dosage form, that delivers the same APIs to the consumer. In one example, the dosage form 96 has an outer perimeter 98 that extends from the first or outer surface 33 to the second surface 45. The first and second surfaces 33 and 45 of the dosage form 96 can be substantially planar, concave, convex, or alternatively shaped as desired. The outer perimeter 98 can be curved such as circular, oval, or alternatively shaped as desired. Voids 32 can be created in the dosage form in the manner described above. For instance, the voids 32 can extend from the first or outer surface 33 toward the second surface 45. The voids 32 can receive API containing liquids in the manner described above. The voids 32 can be subsequently closed in any suitable manner described herein.

It is recognized that the edible substrates 25 of the type described herein can be dosed with any suitable one or more APIs as desired for consumption by specific individuals. For instance, an individual may desire one or more particular APIs, and the particular APIs can be delivered into the voids 32 in the manner described herein. In other examples, a physician can prescribe particular APIs for consumption by a patient. In still other examples, the substrates 25 can be configured as a regimen whereby the substrates 25 vary in quantity and/or constituent components of the APIs vary among the substrates 25. Thus, the substrates 25 can be designed for consumption in a particular order over a predetermined period of time. It is recognized that the substrates can quickly and reliably be dosed in the manner herein to contain any API or combination of APIs as desired.

Referring now to FIG. 11A, it is appreciated that the edible substrate 25 can include one or more layered voids 32. Thus, the edible substrate 25 can be referred to as a layered substrate 25. Each layered void 32 can include first and second different active layers 100 and 102 containing first and second API-containing liquids 31a and 31b that contain first and second APIs, respectively, that are separated by an inactive (not containing an API) partition layer 104. The first active layer 100 can be disposed between the closed end 39, or the outer surface 33, and the second active layer 102. Thus, the first active layer 100 can be referred to as an outer layer, and the second active layer 100 can be referred to as an inner layer. The void 32 can define a first or outer chamber 106 defined between the closed end 39, or the outer surface 33, and the partition layer 104. The first or outer chamber 106 can contain the first active layer 100. The void 32 can define a second or inner chamber 108 defined between the base 37 and the partition layer 104. The second or outer inner 108 can contain the second active layer 102.

As will now be described, the first API can be configured for transmucosal absorption into the bloodstream through a mucosa in the oral cavity, such as the sublingual mucosa or the buccal mucosa, and the second API can be configured for gastrointestinal ingestion. Thus, the first API is primarily bioavailable transmucosally than that through gastrointestinal digestion. As a result, the first API reaches the brain far more quickly as compared to gastrointestinal digestion. Thus, ingestion across each mucosal membrane can allow the first API to have a greater therapeutic effect in a shorter period of time compared to gastrointestinal digestion. Once the first API has been absorbed into the bloodstream through the mucosa in the oral cavity, the edible substrate 25 can be chewed and swallowed. The second API is therefore primarily bioavailable through gastrointestinal ingestion, and is configured to provide a delayed therapeutic effect with respect to the therapeutic effect from the first API. For instance, the therapeutic effect from the second API can take place after the therapeutic effect from the first API has begun, and in some instances after the therapeutic effect from the first API has been completed.

Thus, the user experiences the therapeutic effect of the second API after experiencing the therapeutic effect of the first API. The first API can be configured to provide its therapeutic effect for a duration sufficient to last substantially until the second API delivers its therapeutic effect. Alternatively, the first API can be configured such that its therapeutic effect terminates prior to the delivery of the therapeutic effect of the second API. Alternatively still, the first API can be configured such that its therapeutic effect has a sufficient duration so as to overlap with the therapeutic effect of the second API. Thus, a consumer can select an edible substrate having first and second active layers of first and second APIs, respectively, of their choosing for consumption among a kit of available layered edible substrates 25 that best corresponds to the desired therapeutic effects of the consumer. The API of the first active layer 100 can offer therapeutic effects for relatively short durations that can be optimized based on consumer needs, such as near-term or immediate calmness or other therapeutic effect as desired, whereas the API of the second layer 102 can provide a longer duration of therapeutic effect, and can be optimized for quality of overnight sleep or other therapeutic effect as desired.

With continuing reference to FIG. 11A, the first active layer 22 can be dissolvable by saliva against a sublingual mucosa or a buccal mucosa so as to deliver the first API directly into the bloodstream. In particular, as described above, the open end 39 of the void can be closed with any suitable closure member 79, which can be readily or quickly dissolvable in saliva, for instance when the closure member 79 is placed against a sublingual mucosa and/or a buccal mucosa. Thus, when the closure member 79 dissolves, the first API of the first active layer 100 is exposed to the sublingual or buccal mucosa, and can be transmucosally ingested. In one example, the closure member 79 can be an edible material 82 that is dissolvable by saliva against the sublingual mucosa and/or buccal mucosa. The first API can thus be delivered directly into the bloodstream without undergoing gastrointestinal digestion. The bioavailability of the first API is therefore greater than that through gastrointestinal digestion. Further, the first API reaches the brain far more quickly as compared to gastrointestinal digestion. Thus, ingestion across each mucosal membrane can allow the first API to have a greater therapeutic effect in a shorter period of time compared to gastrointestinal digestion.

Once the first API has been ingested, the edible substrate 25 can be chewed, swallowed, therefore subjecting the second API of the second active layer 102 to gastrointestinal digestion. The gastrointestinal digestion can subject the user to time delayed therapeutic effects of the second API compared to the trans-mucosal ingestion of the first API in the oral cavity. Thus, the first API can provide more immediate therapeutic effects, and the second API can provide therapeutic effects temporally delayed with respect to the effects of the first API. It should be appreciated that the first and second APIs can have different constituent components or different ratios of the same constituent components as desired, depending on the desired effects.

The partition layer 104 is deposited or otherwise applied in the void 32 adjacent the second active layer 102, and separates the first and second active layers 100 and 102 from each other. Thus, the partition layer 104 prevents the first API from traveling from the first active layer 100 to the second active layer 102, and further prevents the second API from traveling from the second active layer 102 to the first active layer 100. The partition layer 104 can be inert or inactive, and thus devoid of API. The partition layer 104 can be constructed such that it is non-dissolvable by saliva against the mucosa, including the buccal mucosa and sublingual mucosa as described above. Alternatively, the partition layer 104 can be sufficiently slowly dissolvable (also referred to as substantially non-dissolvable) by saliva against the mucosal surface in the oral cavity so as to allow time for the consumer to chew and swallow the edible substrate 25 after the closure layer 79 has dissolved, but before the partition layer 104 dissolves. Therefore, the partition layer 104 can be said to be at least substantially non-dissolvable by saliva against the mucosal surface in the oral cavity. As a result, the user has time to masticate and swallow the remaining edible substrate 25 without substantially dissolving the partition layer 104 in saliva after the first API has undergone transmucosal absorption.

The edible substrate 25 can be configured to provide feedback to the user that indicates when the edible substrate 25 is intended to be masticated and swallowed, for instance after the first API has travelled out of the edible substrate 25 for transmucosal absorption. For instance, the first API can have a distinct flavor. When the user experiences the distinct flavor of the first API, the user is alerted that the closure member 79 has been breached or dissolved, and the first API has traveled out of the edible substrate 25. Alternatively or additionally, the partition layer 104 between the first active layer 100 and the second active layer 102 can provide distinct a flavor and/or texture profile. When the consumer experiences the flavor and/or taste profile of the partition layer 104, the consumer is made aware that the first API has traveled out of the edible substrate 25, at which point the user has time to chew and swallow the edible substrate 25 before saliva has breached or dissolved the partition layer 104.

Fabrication of the stacked edible substrate 25 will now be described in one example with initial reference to FIG. 11B. In particular, the second API-containing liquid 31b can be delivered into the pre-formed void 32 in any manner as described above. The second API-containing liquid 31b is thus supported by the base 37 of the void 32. If desired, a drying agent can be applied to the second API-containing liquid 31b as desired to dry the solvent. Alternatively, the solvent of the second API-containing liquid 31b can remain in the void 32. Next, referring to FIG. 11C, the partition layer 104 can be applied to the edible substrate 25 in the void 32 at a location between the second API-containing liquid 31b or the second API and the outer surface 33 or the open end 39. Thus, the second chamber is defined that contains the second API-containing liquid 31b. As described above, the partition layer 104 can either be non-dissolvable or slowly dissolvable in saliva, for instance less dissolvable than the dissolvable closure member 79 (see FIG. 11A). Thus, once the closure member 79 has been dissolved and the first API has been transmucosally ingested, the user has ample time to masticate and swallow the remaining edible substrate 25 before saliva breaches the partition layer 104. Thus, the second API can undergo gastrointestinal digestion. In some examples the partition layer 104 can be made from any suitable gummy material, hard candy material, or other candy material, though it should be appreciated that the partition layer can be made from any suitable edible material such as elastin and/or collagen. In some examples, it may be desirable for the partition layer 104 to have a taste and texture profile that substantially matches that of the edible substrate 25. In other example, the partition layer 104 can be made of the same constituent materials as the closure member 79 but thicker so as to be more resistant to saliva.

Next, referring again to FIG. 11A, the first API-containing liquid 31a can be delivered into the pre-formed void 32, for instance in any manner described above. The first API-containing liquid 31a is thus supported by the partition layer 104 in the void 32. If desired, a drying agent can be applied to the first API-containing liquid 31a as desired to dry the solvent. Alternatively, the solvent of the first API-containing liquid 31a can remain in the void 32. The closure member 79 can then be applied to the opening 39, thereby closing the void 32 at the first or outer surface 33 in any suitable manner described herein. The closure member 79 can be dissolvable against a mucosal surface in the oral cavity as described above.

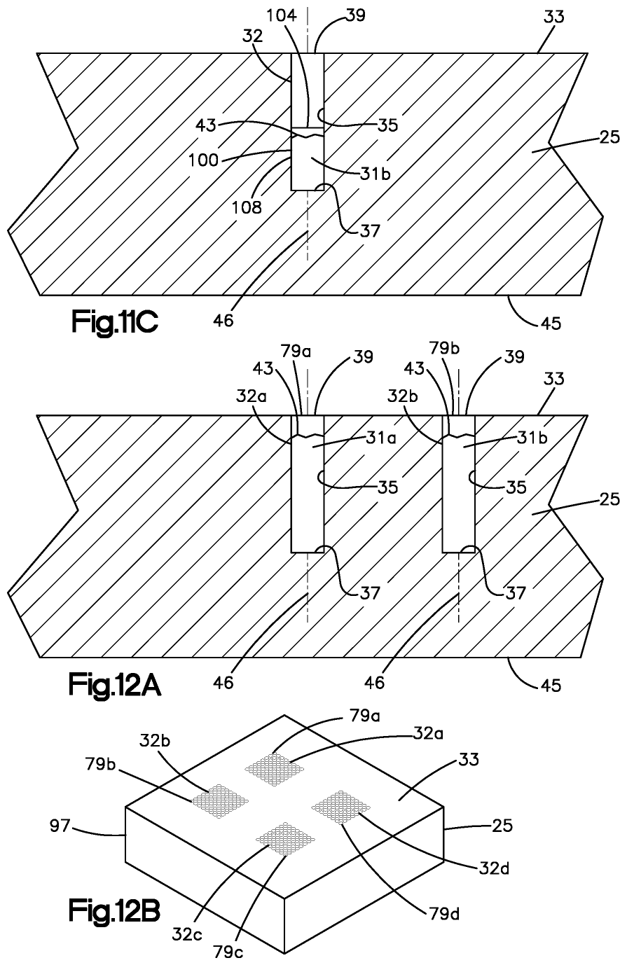

Referring now to FIG. 12A, the edible substrate 25 can be alternatively constructed such that the first API-containing liquid 31a or first API can be delivered directly through the bloodstream through a mucosal surface in the oral cavity, and the second API-containing liquid 31b or a second API is configured for gastrointestinal digestion. In particular, the voids 32 can include at least one first void 32a and at least one second void 32b. The first API-containing liquid 31a can be delivered into the first void 32a, for instance in any manner described above, such that the first API-containing liquid 31a extends from the base toward the opening 39 at the first or outer surface 33 along a portion up to a majority such as a substantial entirety of the first void 32a. If desired, a drying agent can be applied to the first API-containing liquid 31a as desired to dry the solvent. Alternatively, the solvent of the first API-containing liquid 31a can remain in the first void 32a. Similarly, the second API-containing liquid 31b can be delivered into the second void 32b, for instance in any manner described above, such that the second API-containing liquid 31b extends from the base toward the opening 39 at the first or outer surface along a portion up to a majority such as a substantial entirety of the second void 32b. If desired, a drying agent can be applied to the second API-containing liquid 31b as desired to dry the solvent. Alternatively, the solvent of the second API-containing liquid 31b can remain in the second void 32b. Whether the solvents of the first and second API-containing liquids 31a and 31b are dissolved or not, the first and second voids 32a and 32b can be said to include the first and second APIs, respectively.

The first open ends 39 of the second voids 32a and 32b can be closed with first and second closure members 79a and 79b as desired. The first closure members 79a can be readily or quickly dissolvable by saliva against a mucosal surface. For instance, the first closure member 79a can be defined by an edible material 82 that is dissolvable by saliva against a sublingual mucosa and/or a buccal mucosa so as to deliver the first API transmucosally directly into the bloodstream. Therefore, the first API can thus be delivered directly into the bloodstream without undergoing gastrointestinal digestion. The bioavailability of the first API is therefore greater than that through gastrointestinal digestion. Further, the first API reaches the brain far more quickly as compared to gastrointestinal digestion. Thus, ingestion across each mucosal membrane can allow the first API to have a greater therapeutic effect in a shorter period of time compared to gastrointestinal digestion.

The second closure member 79b can be substantially non-dissolvable in saliva. Alternatively, the second closure member 79b can be less dissolvable in saliva than the first closure member 79a. In particular, the second closure member 79b can be sufficiently slowly dissolvable (also referred to as substantially non-dissolvable) by saliva against the mucosal surface in the oral cavity so as to allow time for the consumer to chew and swallow the edible substrate 25 after the first closure layer 79a has dissolved in saliva, but before the second closure layer 79 is breached or otherwise dissolved in saliva. Therefore, the second closure member 79b can be said to be at least substantially non-dissolvable by saliva against the mucosal surface in the oral cavity. One example of a non-dissolvable or slowly dissolvable second closure member 79b can be configured as a closed seam 86 (see FIG. 6C) that can be produced by bonding different regions of the edible substrate 25 to each other. Another example of a non-dissolvable or slowly dissolvable second closure member 79b can be configured as an edible material 82 that is substantially non-dissolvable in saliva include any suitable gummy material, hard candy material, or other candy material, though it should be appreciated that the second closure member 79b layer can be made from any suitable edible material such as meat gristle. In some examples, it may be desirable for the second closure member 79b to have a taste and texture profile that substantially matches that of the edible substrate 25. In other example, the partition second closure member 79b can be made of the same constituent materials as the first closure member 79b, but thicker so as to be more resistant to saliva. Because the second closure member 79b is less dissolvable in saliva than the first closure member 79a, the second closure member 79b remains after the first closure member 79a has been breached and the first API has been transmucosally ingested. Therefore, once the first API has been ingested, user has time to masticate and swallow the edible substrate 25, swallowed, thereby subjecting the second API of the second active layer 102 to gastrointestinal digestion.

The edible substrate 25 can be configured to provide feedback to the user that indicates when the edible substrate 25 is intended to be masticated and swallowed, for instance after the first API has travelled out of the edible substrate 25 for transmucosal absorption. For instance, the first API can have a distinct flavor. When the user experiences the distinct flavor of the first API, the user is alerted that the first closure member 79a has been breached or dissolved, and the first API has traveled out of the edible substrate 25. Alternatively or additionally, the second closure member 79b can provide distinct a flavor and/or texture profile. When the consumer experiences the flavor and/or taste profile of the second closure member 79b, the consumer is made aware that the first API has traveled out of the edible substrate 25, at which point the user has time to chew and swallow the edible substrate 25 before saliva has breached or dissolved the second closure member 79b.

The gastrointestinal digestion of the second API can subject the user to time delayed therapeutic effects of the second API compared to the trans-mucosal ingestion of the first API in the oral cavity. Thus, the first API can provide more immediate therapeutic effects, and the second API can provide therapeutic effects temporally delayed with respect to the effects of the first API. It should be appreciated that the first and second APIs can have different constituent components or different ratios of the same constituent components as desired, depending on the desired effects.

Referring to FIG. 12B, the edible substrate 25 can include one or more groups that each includes at least one void. For instance, the edible substrate 25 is shown as including a plurality of groups of voids including first, second, third, and fourth groups of voids 32a, 32b, 32c, and 32d of voids, respectively. The groups of voids 32a-32b can contain first, second, third, and fourth APIs, respectively, and can be closed by respective closure members 79a, 79b, 79c, and 79d, respectively. The groups of voids 32a-32d can be located at different respective locations or zones of the edible substrate 25. Alternatively, the groups of voids 32a-32d can be interspersed throughout the edible substrate 25. The voids of at least one of the groups can be accessible in saliva more readily than the voids of at least one other of the groups. For instance, the first closure members 79a of the first group of voids 32a can be more readily or quickly dissolvable in saliva than the closure members 79b-79d of the other groups of voids 32b-32d. The second closure members 79b. The second closure members 79b of the second group of voids 32b can be more readily or quickly dissolvable in saliva than the closure members 79c-79d of the third and fourth groups of voids 32c-32d. The third closure members 79c of the third group of voids 32c can be more readily or quickly dissolvable in saliva than the closure members 79d of the fourth group of voids 32d. In one example, the closure member of a final group of voids, which can be defined by the fourth closure member 79d of the fourth group of voids 32d, can be substantially non-dissolvable in saliva. Therefore, the API in the voids of the final group of voids can be configured for gastrointestinal absorption.

Thus, when the edible substrate 25 is disposed in the oral cavity, saliva can sequentially dissolve the closure members of a plurality of the groups of voids, for instance the closure members 79a-79c of voids 32a-32c. For each closure member of the plurality of the groups of voids, once the saliva has dissolved the closure member, the API disposed in the respective void can be transmucosally absorbed. Once the API has travelled out of the voids of a final group of voids to be dissolved in saliva (which can be defined by the third group of voids 32c), the user can then masticate and swallow the edible substrate. Therefore, the API disposed in the remaining or final group of voids, such as the fourth group of voids 32d, can be gastrointestinally absorbed.

The closure members that are dissolvable in saliva can be tuned to dissolve at different approximate points in time after exposure to saliva as desired. For instance, the first closure members 79a can be breached or dissolved, which allows the first API to travel out of the first group of voids 32a for transmucosal absorption. The second closure member 79b can be breached or dissolved in saliva after the first API has begun undergoing transmucosal absorption, and in some instances after the first API has completed transmucosal absorption. When the second closure members 79b are breached or dissolved, the second API travels out of the second group of voids 32b for transmucosal absorption. Similarly, the third closure member 79c can be breached or dissolved in saliva after the second API has begun undergoing transmucosal absorption, and in some instances after the second API has completed transmucosal absorption. When the third closure members 79c are breached or dissolved, the third API travels out of the third group of voids 32c for transmucosal absorption. It should be appreciated that the edible substrate 25 can include any number of groups of voids as desired whose respective contained APIs are configured for sequential transmucosal absorption.

Once a final one of the APIs configured for transmucosal absorption has travelled out of the edible substrate 25, the edible substrate 25 can then be masticated and swallowed, thereby subjecting the remaining API disposed in respective remaining one or more groups of voids to gastrointestinal absorption. The final API configured for transmucosal absorption can have a distinct flavor profile. Therefore, when the user senses the distinct flavor profile, the user is alerted that the final API configured for transmucosal absorption has travelled out of the edible substrate 25. The user, in response, can then masticate and swallow the edible substrate 25. The final group of voids 32d whose closure member 79d is substantially non-dissolvable. Accordingly, the user can masticate and swallow the edible substrate 25 prior to the final closure member 79d being breached or dissolved by saliva. It should be appreciated that the at least one API disposed in each group of voids can be different than the at least one API disposed in the other groups of voids. Alternatively, the API of the plurality of groups of voids can be the same, so as to allow for a sequential quick-hitting but sustained therapeutic effect. The therapeutic effect of the API of each of the plurality of groups of vaults can be delivered sequentially and at different points in time as desired, all of which can be controlled for instance by tuning the dissolvability of the closure member in saliva. It should be groups of voids 32a-32d can be differently sized and/or contain different volumes of respective API-containing liquids. Alternatively, the groups of voids 32a-32d can be sized and shaped substantially identical to each other, and can contain approximately the same volume of API.

It should be noted that the illustrations and discussions of the embodiments and examples shown in the figures are for exemplary purposes only and should not be construed limiting the disclosure. One skilled in the art will appreciate that the present disclosure contemplates a range of possible modifications of the various aspects, embodiments and examples described herein. Additionally, it should be understood that the concepts described above with the above-described embodiments and examples may be employed alone or in combination with any of the other embodiments and examples described above. It should further be appreciated that the various alternatives described above with respect to one illustrated embodiment can apply to all other embodiments and examples described herein, unless otherwise indicated. Reference is therefore made to the claims.

What is claimed:

1. A method of delivering an active pharmaceutical ingredient (API) to an edible substrate, the method comprising the steps of:
positioning the edible substrate such that a pre-formed void that extends into the edible substrate is in operable alignment with a dosing head; and
after the positioning step, delivering a volume of at least one API-containing liquid from the dosing head into the void.

2. The method of claim 1, wherein the positioning step comprises moving at least one of the edible substrate and the dosing head such that the dosing head is in alignment with the void.

3. The method of claim 1, wherein the delivering step comprises delivering the API-containing liquid as a plurality of microdroplets, and the delivering step comprises delivering the plurality of microdroplets sequentially and individually from the dosing head into the void.

4. The method of claim 1, wherein the pre-formed void extends into but not through the edible substrate.

5. The method of claim 1, wherein the pre-formed void comprises a plurality of voids, the pre-formed void is a first pre-formed void, the volume of API-containing liquid is a first volume of API-containing liquid, and the dosing head is a first dosing head, the method further comprising the steps of:
delivering a second volume of API-containing liquid from a second dosing head into a second one of the plurality of voids.

6. The method of claim 5, wherein the API of the second volume of API-containing liquid has a different active ingredient than the API of the first volume of API-containing liquid.

7. The method of claim 1, further comprising the step of creating the pre-formed void.

8. The method of claim 7, wherein the step of creating comprises ablating the edible substrate.

9. The method of claim 8, wherein the ablating step is performed with at least one laser.

10. The method of claim 7, wherein the step of creating the preformed void comprises driving a shaft into the edible substrate.

11. The method of claim 10, wherein the shaft compresses the edible substrate so as to create the void.

12. The method of claim 10, wherein the shaft is a heated shaft that selectively at least partially melts the edible substrate so as to create the void.

13. The method of claim 1, further comprising, after the delivering step, the step of closing the void.

14. The method of claim 13, wherein the void extends into a surface of the edible substrate, and the closing step comprises displacing the edible substrate at the surface so as to close the void.

15. The method of claim 13, wherein the closing step comprises delivering an edible material to the edible substrate at least at an open end of the void, wherein the edible material closes the open end.

16. The method of claim 1, wherein the void extends into the edible substrate along a central axis, and the void defines a cross-sectional area along a direction perpendicular to the central axis that is in the range of 5 millionths of an inch up to approximately 0.25 inch.

17. The method of claim 1, wherein the edible substrate is substantially non-porous such that the API-containing liquid remains in the void after the delivering step.

18. The method of claim 1, wherein the edible substrate is porous such that the API-containing liquid travels from the void into a surrounding region of the edible substrate.

19. The method of claim 1, further comprising applying a barrier to at least one of an internal surface and a base that that combine to define the void, wherein the barrier is substantially non-porous so as to prevent the API disposed in the void from traveling through the internal surface into an adjacent region of the edible substrate.

20. The method of claim 1, wherein the edible substrate comprises a food product.

21. The method of claim 1, wherein the edible substrate comprises a dosage form.

22. The method of claim 1, comprising the step of configuring a first at least one API for transmucosal absorption, and configuring at least one second API for gastrointestinal absorption, and delivering the at least one first API and the at least one second API into the void such that the at least one first API and the at least one second API are separated from each other in the void.
